SEQUENCE LISTING (28 sequences)

SEQ ID NO: 1 - protein, 94 aa, c. autoethanogenum
MKIRPLGDRVVIKKLEAEETTKSGIVLPGSAKEKPQEAEVVAVGIGGTVDGKEVKMEVKVGDKVLFSKYAGNEVKIDAQEYTILKQDDILAIIE

SEQ ID NO: 2 - protein, 544 aa, c. autoethanogenum
MAKSILFGEDARKSMQEGVNKLANAVKVTLGPKGRNVVLDKKFGSPLITNDGVTIAKEIELEDPYENMGAQLVKEVATKTNDVAGDGTTTATLLAQAIIREGLKNVTAGANPMLIRQGIKMAVDKAVEEIKKVSTTVKGKEDIARIAAISASDEEIGKLIADAMEKVGNEGVITVEESKTMGTELDVVEGMQFDRGYLSPYMVTDSEKMEAAIEDPYILITDKKISNIQDILPLLEKIVQQGKKLLIIAEDVEGEALATLVVNKLRGTFTCVAVKAPGFGDRRKEMLQDIAILTGGQVISEELGRDLKEAELEDLGRAESVKIDKENTTIVNGRGDKKAIADRVSQIKVQIEETTSDFDKEKLQERLAKLAGGVAVVKVGAATETELKEKKLRIEDALAATKAGVEEGMGPGGGTAYINAIPEVEKLTSDVPDVKVGIDIIRKALEEPVRQIASNAGVEGSVIIQKVRNSEIGVGYDALKGEYVNMVEKGIVDPTKVTRSALQNAASVAATFLTTEAAVADIPEKAPAGPAAGAPGMGGMEGMY

SEQ ID NO: 3 - DNA, 285 bp, c. autoethanogenum
atgaaaattagaccacttggagacagagttgtaattaaaaaattagaagctgaggaaactacgaagagcggtattgttttaccaggaagtgctaaagaaaaaccacaagaagcagaagttgtggcagtaggaattggtggaacagtagatggaaaagaagttaaaatggaagtaaaagtaggagataaggtattattctccaaatatgctggaaatgaagtaaaaatagatgcacaagagtacactattttaaaacaggacgacatattagctataatcgagtag

SEQ ID NO: 4 - DNA, 1635 bp, c. auotethanogenum
atggcaaaaagtattttatttggtgaagatgcaagaaaatcaatgcaagaaggtgtaaataagctagcaaatgcagtaaaggttacacttggacctaagggaagaaatgtagtacttgataagaaatttggttcaccgcttattacaaatgacggtgttacaatagcaaaggaaatagaattagaagatccatatgaaaacatgggagcacaacttgtaaaagaagttgctacaaagacaaatgatgtagctggagatggaacaactacagctactttacttgctcaagcaataataagagaaggattaaaaaatgttacagctggagcaaatccaatgcttataagacaaggtataaagatggctgtagataaagctgtagaagaaataaaaaaagtttcaacaactgtaaagggaaaagaagatatagcaagaattgcagctatatcagcttctgatgaagaaataggtaaattaatagctgatgccatggaaaaggtaggtaacgaaggtgtcataactgttgaagagtcaaaaactatgggaactgagttagatgtagttgaaggtatgcagtttgacagaggttatttaagtccatatatggttactgattcagaaaaaatggaagctgcaatagaagatccatatatattaataacagacaagaagatatcaaatattcaagatatattaccattacttgagaaaatagttcaacaaggaaagaagttacttataatagctgaagatgtagaaggagaagcacttgcaactttagttgtaaataagttaagaggaacatttacttgtgtagcagtaaaggcacctggatttggtgacagaagaaaagaaatgcttcaggatatagcaatacttactggaggacaggtaatatcagaagaattgggaagagacttaaaagaagctgaattagaggatttaggaagagctgaatctgtaaagatagataaagaaaatactactatagtaaatggacgaggagataagaaagctatagcagatagagtatcccagattaaggttcaaatagaagaaactacttcagattttgataaagaaaaacttcaagaaagacttgcaaaacttgcaggtggagtagctgtagtaaaagttggagcagcaactgaaactgaattaaaagagaaaaaattaagaatagaagatgctttagcagctacaaaagcaggtgttgaagaaggtatgggaccaggaggcggaactgcttatataaatgcaattccagaagttgaaaaattaacttcagatgtaccggatgtaaaagttggtatagacataataagaaaagcattggaagaaccagttagacaaatagcaagcaatgctggtgttgaaggttcagtaataatccaaaaagttagaaatagtgaaattggtgttggatacgatgcattaaaaggcgaatatgtaaacatggtagaaaagggtatagtagacccaactaaggttacaagatcagcacttcaaaatgcagcatccgtagcagctacattcttaactacagaagcagcagttgcagatattccagaaaaagcacctgcaggtccagcagcaggagcaccaggaatgggcggaatggaaggaatgtactaa

SEQ ID NO: 5 - DNA, 479 bp, c. autoethanogenum
ggccgcaaaatagttgataataatgcagagttataaacaaaggtgaaaagcattacttgtattcttttttatatattattataaattaaaatgaagctgtattagaaaaaatacacacctgtaatataaaattttaaattaatttttaattttttcaaaatgtattttacatgtttagaattttgatgtatattaaaatagtagaatacataagatacttaatttaattaaagatagttaagtacttttcaatgtgcttttttagatgtttaatacaaatctttaattgtaaaagaaatgctgtactatttactgtactagtgacgggattaaactgtattaattataaataaaaaataagtacagttgtttaaaattatattttgtattaaatctaatagtacgatgtaagttattttatactattgctagtttaataaaaagatttaattatatacttgaaaaggagaggaatccat

SEQ ID NO: 6 - DNA, 28 bp, Artificial sequence
  Synthetic primer
gggttcatatgaaaattagaccacttgg

SEQ ID NO: 7 - DNA, 30 bp, Artificial sequence
  Synthetic primer
tcccatgttttcataaggatcttctaattc

SEQ ID NO: 8 - DNA, 30 bp, Artificial sequence
  Synthetic primer
attagaagatccttatgaaaacatgggagc

SEQ ID NO: 9 - DNA, 30 bp, Artificial sequence
  Synthetic primer
cttagaattccttttgaattagtacattcc

SEQ ID NO: 10 - DNA, 30 bp, Artificial sequence
  Synthetic primer
aagcggccgcaaaatagttgataataatgc

SEQ ID NO: 11 - DNA, 30 bp, Artificial sequence
  Synthetic primer
tacgcatatgaattcctctccttttcaagc

SEQ ID NO: 12 - DNA, 1978 bp, c. autoethanogenum
atgaaaattagaccacttggagacagagttgtaattaaaaaattagaagctgaggaaactacgaagagcggtattgttttaccaggaagtgctaaagaaaaaccacaagaagcagaagttgtggcagtaggaattggtggaacagtagatggaaaagaagttaaaatggaagtaaaagtaggagataaggtattattctccaaatatgctggaaatgaagtaaaaatagatgcacaagagtacactattttaaaacaggacgacatattagctataatcgagtagttaattgaaaaagaaaaataagtatctatataacggttagttgtaaggagggttttttatggcaaaaagtattttatttggtgaagatgcaagaaaatcaatgcaagaaggtgtaaataagctagcaaatgcagtaaaggttacacttggacctaagggaagaaatgtagtacttgataagaaatttggttcaccgcttattacaaatgacggtgttacaatagcaaaggaaatagaattagaagatccttatgaaaacatgggagcacaacttgtaaaagaagttgctacaaagacaaatgatgtagctggagatggaacaactacagctactttacttgctcaagcaataataagagaaggattaaaaaatgttacagctggagcaaatccaatgcttataagacaaggtataaagatggctgtagataaagctgtagaagaaataaaaaaagtttcaacaactgtaaagggaaaagaagatatagcaagaattgcagctatatcagcttctgatgaagaaataggtaaattaatagctgatgccatggaaaaggtaggtaacgaaggtgtcataactgttgaagagtcaaaaactatgggaactgagttagatgtagttgaaggtatgcagtttgacagaggttatttaagtccatatatggttactgattcagaaaaaatggaagctgcaatagaagatccatatatattaataacagacaagaagatatcaaatattcaagatatattaccattacttgagaaaatagttcaacaaggaaagaagttacttataatagctgaagatgtagaaggagaagcacttgcaactttagttgtaaataagttaagaggaacatttacttgtgtagcagtaaaggcacctggatttggtgacagaagaaaagaaatgcttcaggatatagcaatacttactggaggacaggtaatatcagaagaattgggaagagacttaaaagaagctgaattagaggatttaggaagagctgaatctgtaaagatagataaagaaaatactactatagtaaatggacgaggagataagaaagctatagcagatagagtatcccagattaaggttcaaatagaagaaactacttcagattttgataaagaaaaacttcaagaaagacttgcaaaacttgcaggtggagtagctgtagtaaaagttggagcagcaactgaaactgaattaaaagagaaaaaattaagaatagaagatgctttagcagctacaaaagcaggtgttgaagaaggtatgggaccaggaggcggaactgcttatataaatgcaattccagaagttgaaaaattaacttcagatgtaccggatgtaaaagttggtatagacataataagaaaagcattggaagaaccagttagacaaatagcaagcaatgctggtgttgaaggttcagtaataatccaaaaagttagaaatagtgaaattggtgttggatacgatgcattaaaaggcgaatatgtaaacatggtagaaaagggtatagtagacccaactaaggttacaagatcagcacttcaaaatgcagcatccgtagcagctacattcttaactacagaagcagcagttgcagatattccagaaaaagcacctgcaggtccagcagcaggagcaccaggaatgggcggaatggaaggaatgtactaa

SEQ ID NO: 13 - DNA, 16 bp, Artificial sequence
  Synthetic primer
gtaaaacgacggccag

SEQ ID NO: 14 - DNA, 17 bp, Artificial sequence
  Synthetic primer
caggaaacagctatgac

SEQ ID NO: 15 - DNA, 2963 bp, e. coli
cctgcaggataaaaaaattgtagataaattttataaaatagttttatctacaatttttttatcaggaaacagctatgaccgcggccgctgtatccatatgaccatgattacgaattcgagctcggtacccggggatcctctagagtcgacgtcacgcgtccatggagatctcgaggcctgcagacatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctagcataaaaataagaagcctgcatttgcaggcttcttatttttatggcgcgccgcattcacttcttttctatataaatatgagcgaagcgaataagcgtcggaaaagcagcaaaaagtttcctttttgctgttggagcatgggggttcagggggtgcagtatctgacgtcaatgccgagcgaaagcgagccgaagggtagcatttacgttagataaccccctgatatgctccgacgctttatatagaaaagaagattcaactaggtaaaatcttaatataggttgagatgataaggtttataaggaatttgtttgttctaatttttcactcattttgttctaatttcttttaacaaatgttcttttttttttagaacagttatgatatagttagaatagtttaaaataaggagtgagaaaaagatgaaagaaagatatggaacagtctataaaggctctcagaggctcatagacgaagaaagtggagaagtcatagaggtagacaagttataccgtaaacaaacgtctggtaacttcgtaaaggcatatatagtgcaattaataagtatgttagatatgattggcggaaaaaaacttaaaatcgttaactatatcctagataatgtccacttaagtaacaatacaatgatagctacaacaagagaaatagcaaaagctacaggaacaagtctacaaacagtaataacaacacttaaaatcttagaagaaggaaatattataaaaagaaaaactggagtattaatgttaaaccctgaactactaatgagaggcgacgaccaaaaacaaaaatacctcttactcgaatttgggaactttgagcaagaggcaaatgaaatagattgacctcccaataacaccacgtagttattgggaggtcaatctatgaaatgcgattaagggccggccagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaagtttaaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccc

SEQ ID NO: 16 - DNA, 5935 bp, e. coli
cctgcaggataaaaaaattgtagataaattttataaaatagttttatctacaatttttttatcaggaaacagctatgaccgcggccgctgtatccatatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaaacgcgtccatggagatctcgaggcctgcagacatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctagcataaaaataagaagcctgcatttgcaggcttcttatttttatggcgcgccgttctgaatccttagctaatggttcaacaggtaactatgacgaagatagcaccctggataagtctgtaatggattctaaggcatttaatgaagacgtgtatataaaatgtgctaatgaaaaagaaaatgcgttaaaagagcctaaaatgagttcaaatggttttgaaattgattggtagtttaatttaatatattttttctattggctatctcgatacctatagaatcttctgttcacttttgtttttgaaatataaaaaggggctttttagccccttttttttaaaactccggaggagtttcttcattcttgatactatacgtaactattttcgatttgacttcattgtcaattaagctagtaaaatcaatggttaaaaaacaaaaaacttgcatttttctacctagtaatttataattttaagtgtcgagtttaaaagtataatttaccaggaaaggagcaagttttttaataaggaaaaatttttccttttaaaattctatttcgttatatgactaattataatcaaaaaaatgaaaataaacaagaggtaaaaactgctttagagaaatgtactgataaaaaaagaaaaaatcctagatttacgtcatacatagcacctttaactactaagaaaaatattgaaaggacttccacttgtggagattatttgtttatgttgagtgatgcagacttagaacattttaaattacataaaggtaatttttgcggtaatagattttgtccaatgtgtagttggcgacttgcttgtaaggatagtttagaaatatctattcttatggagcatttaagaaaagaagaaaataaagagtttatatttttaactcttacaactccaaatgtaaaaagttatgatcttaattattctattaaacaatataataaatcttttaaaaaattaatggagcgtaaggaagttaaggatataactaaaggttatataagaaaattagaagtaacttaccaaaaggaaaaatacataacaaaggatttatggaaaataaaaaaagattattatcaaaaaaaaggacttgaaattggtgatttagaacctaattttgatacttataatcctcattttcatgtagttattgcagttaataaaagttattttacagataaaaattattatataaatcgagaaagatggttggaattatggaagtttgctactaaggatgattctataactcaagttgatgttagaaaagcaaaaattaatgattataaagaggtttacgaacttgcgaaatattcagctaaagacactgattatttaatatcgaggccagtatttgaaattttttataaagcattaaaaggcaagcaggtattagtttttagtggattttttaaagatgcacacaaattgtacaagcaaggaaaacttgatgtttataaaaagaaagatgaaattaaatatgtctatatagtttattataattggtgcaaaaaacaatatgaaaaaactagaataagggaacttacggaagatgaaaaagaagaattaaatcaagatttaatagatgaaatagaaatagattaaagtgtaactatactttatatatatatgattaaaaaaataaaaaacaacagcctattaggttgttgttttttattttctttattaatttttttaatttttagtttttagttcttttttaaaataagtttcagcctctttttcaatattttttaaagaaggagtatttgcatgaattgccttttttctaacagacttaggaaatattttaacagtatcttcttgcgccggtgattttggaacttcataacttactaatttataattattattttcttttttaattgtaacagttgcaaaagaagctgaacctgttccttcaactagtttatcatcttcaatataatattcttgacctatatagtataaatatatttttattatatttttacttttttctgaatctattattttataatcataaaaagttttaccaccaaaagaaggttgtactccttctggtccaacatatttttttactatattatctaaataatttttgggaactggtgttgtaatttgattaatcgaacaaccagttatacttaaaggaattataactataaaaatatataggattatctttttaaatttcattattggcctcctttttattaaatttatgttaccataaaaaggacataacgggaatatgtagaatatttttaatgtagacaaaattttacataaatataaagaaaggaagtgtttgtttaaattttatagcaaactatcaaaaattagggggataaaaatttatgaaaaaaaggttttcgatgttatttttatgtttaactttaatagtttgtggtttatttacaaattcggccggccgaagcaaacttaagagtgtgttgatagtgcagtatcttaaaattttgtataataggaattgaagttaaattagatgctaaaaatttgtaattaagaaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtgtttaaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgatcgggccc

SEQ ID NO: 17 - DNA, 5512 bp, Artificial sequence
  synthetic plasmid
ggccgcaaaatagttgataataatgcagagttataaacaaaggtgaaaagcattacttgtattcttttttatatattattataaattaaaatgaagctgtattagaaaaaatacacacctgtaatataaaattttaaattaatttttaattttttcaaaatgtattttacatgtttagaattttgatgtatattaaaatagtagaatacataagatacttaatttaattaaagatagttaagtacttttcaatgtgcttttttagatgtttaatacaaatctttaattgtaaaagaaatgctgtactatttactgtactagtgacgggattaaactgtattaattataaataaaaaataagtacagttgtttaaaattatattttgtattaaatctaatagtacgatgtaagttattttatactattgctagtttaataaaaagatttaattatatacttgaaaaggagaggaatccatatgaaaattagaccacttggagacagagttgtaattaaaaaattagaagctgaggaaactacgaagagcggtattgttttaccaggaagtgctaaagaaaaaccacaagaagcagaagttgtggcagtaggaattggtggaacagtagatggaaaagaagttaaaatggaagtaaaagtaggagataaggtattattctccaaatatgctggaaatgaagtaaaaatagatgcacaagagtacactattttaaaacaggacgacatattagctataatcgagtagttaattgaaaaagaaaaataagtatctatataacggttagttgtaaggagggttttttatggcaaaaagtattttatttggtgaagatgcaagaaaatcaatgcaagaaggtgtaaataagctagcaaatgcagtaaaggttacacttggacctaagggaagaaatgtagtacttgataagaaatttggttcaccgcttattacaaatgacggtgttacaatagcaaaggaaatagaattagaagatccttatgaaaacatgggagcacaacttgtaaaagaagttgctacaaagacaaatgatgtagctggagatggaacaactacagctactttacttgctcaagcaataataagagaaggattaaaaaatgttacagctggagcaaatccaatgcttataagacaaggtataaagatggctgtagataaagctgtagaagaaataaaaaaagtttcaacaactgtaaagggaaaagaagatatagcaagaattgcagctatatcagcttctgatgaagaaataggtaaattaatagctgatgccatggaaaaggtaggtaacgaaggtgtcataactgttgaagagtcaaaaactatgggaactgagttagatgtagttgaaggtatgcagtttgacagaggttatttaagtccatatatggttactgattcagaaaaaatggaagctgcaatagaagatccatatatattaataacagacaagaagatatcaaatattcaagatatattaccattacttgagaaaatagttcaacaaggaaagaagttacttataatagctgaagatgtagaaggagaagcacttgcaactttagttgtaaataagttaagaggaacatttacttgtgtagcagtaaaggcacctggatttggtgacagaagaaaagaaatgcttcaggatatagcaatacttactggaggacaggtaatatcagaagaattgggaagagacttaaaagaagctgaattagaggatttaggaagagctgaatctgtaaagatagataaagaaaatactactatagtaaatggacgaggagataagaaagctatagcagatagagtatcccagattaaggttcaaatagaagaaactacttcagattttgataaagaaaaacttcaagaaagacttgcaaaacttgcaggtggagtagctgtagtaaaagttggagcagcaactgaaactgaattaaaagagaaaaaattaagaatagaagatgctttagcagctacaaaagcaggtgttgaagaaggtatgggaccaggaggcggaactgcttatataaatgcaattccagaagttgaaaaattaacttcagatgtaccggatgtaaaagttggtatagacataataagaaaagcattggaagaaccagttagacaaatagcaagcaatgctggtgttgaaggttcagtaataatccaaaaagttagaaatagtgaaattggtgttggatacgatgcattaaaaggcgaatatgtaaacatggtagaaaagggtatagtagacccaactaaggttacaagatcagcacttcaaaatgcagcatccgtagcagctacattcttaactacagaagcagcagttgcagatattccagaaaaagcacctgcaggtccagcagcaggagcaccaggaatgggcggaatggaaggaatgtactaattcaaaaggaattcgagctcggtacccggggatcctctagagtcgacgtcacgcgtccatggagatctcgaggcctgcagacatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctagcataaaaataagaagcctgcatttgcaggcttcttatttttatggcgcgccgcattcacttcttttctatataaatatgagcgaagcgaataagcgtcggaaaagcagcaaaaagtttcctttttgctgttggagcatgggggttcagggggtgcagtatctgacgtcaatgccgagcgaaagcgagccgaagggtagcatttacgttagataaccccctgatatgctccgacgctttatatagaaaagaagattcaactaggtaaaatcttaatataggttgagatgataaggtttataaggaatttgtttgttctaatttttcactcattttgttctaatttcttttaacaaatgttcttttttttttagaacagttatgatatagttagaatagtttaaaataaggagtgagaaaaagatgaaagaaagatatggaacagtctataaaggctctcagaggctcatagacgaagaaagtggagaagtcatagaggtagacaagttataccgtaaacaaacgtctggtaacttcgtaaaggcatatatagtgcaattaataagtatgttagatatgattggcggaaaaaaacttaaaatcgttaactatatcctagataatgtccacttaagtaacaatacaatgatagctacaacaagagaaatagcaaaagctacaggaacaagtctacaaacagtaataacaacacttaaaatcttagaagaaggaaatattataaaaagaaaaactggagtattaatgttaaaccctgaactactaatgagaggcgacgaccaaaaacaaaaatacctcttactcgaatttgggaactttgagcaagaggcaaatgaaatagattgacctcccaataacaccacgtagttattgggaggtcaatctatgaaatgcgattaagggccggccgaagcaaacttaagagtgtgttgatagtgcagtatcttaaaattttgtataataggaattgaagttaaattagatgctaaaaatttgtaattaagaaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtgtttaaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcaggataaaaaaattgtagataaattttataaaatagttttatctacaatttttttatcaggaaacagctatgaccgc

SEQ ID NO: 18 - protein, 601 aa, Artificial sequence
  synthetic protein
MFPCNAYIEYGDKNMNSFIEDVEQIYNFIKKNIDVEEKMHFIETYKQKSNMKKEISFSEEYYKQKIMNGKNGVVYTPPEMAAFMVKNLINVNDVIGNPFIKIIDPSCGSGNLICKCFLYLNRIFIKNIEVINSKNNLNLKLEDISYHIVRNNLFGFDIDETAIKVLKIDLFLISNQFSEKNFQVKDFLVENIDRKYDVFIGNPPYIGHKSVDSSYSYVLRKIYGSIYRDKGDISYCFFQKSLKCLKEGGKLVFVTSRYFCESCSGKELRKFLIENTSIYKIIDFYGIRPFKRVGIDPMIIFLVRTKNWNNNIEIIRPNKIEKNEKNKFLDSLFLDKSEKCKKFSISQKSINNDGWVFVDEVEKNIIDKIKEKSKFILKDICHSCQGIITGCDRAFIVDRDIINSRKIELRLIKPWIKSSHIRKNEVIKGEKFIIYSNLIENETECPNAIKYIEQYKKRLMERRECKKGTRKWYELQWGRKPEIFEEKKIVFPYKSCDNRFALDKGSYFSADIYSLVLKKNVPFTYEILLNILNSPLYEFYFKTFAKKLGENLYEYYPNNLMKLCIPSIDFGGENNIEKKLYDFFGLTDKEIEIVEKIKDNC

SEQ ID NO: 19 - DNA, 4709 bp, Artificial sequence
  synthetic plasmid
gtttgccacctgacgtctaagaaaaggaatattcagcaatttgcccgtgccgaagaaaggcccacccgtgaaggtgagccagtgagttgattgctacgtaattagttagttagcccttagtgactcgtaatacgactcactatagggctcgaggcggccgcgcaacgcaattaatgtgagttagctcactcattaggcaccccaggctttacactttatgcttccggctcgtatgttgtgtggaattgtgagcggataacaatttcacacaggaaacacatatgtttccgtgcaatgcctatatcgaatatggtgataaaaatatgaacagctttatcgaagatgtggaacagatctacaacttcattaaaaagaacattgatgtggaagaaaagatgcatttcattgaaacctataaacagaaaagcaacatgaagaaagagattagctttagcgaagaatactataaacagaagattatgaacggcaaaaatggcgttgtgtacaccccgccggaaatggcggcctttatggttaaaaatctgatcaacgttaacgatgttattggcaatccgtttattaaaatcattgacccgagctgcggtagcggcaatctgatttgcaaatgttttctgtatctgaatcgcatctttattaagaacattgaggtgattaacagcaaaaataacctgaatctgaaactggaagacatcagctaccacatcgttcgcaacaatctgtttggcttcgatattgacgaaaccgcgatcaaagtgctgaaaattgatctgtttctgatcagcaaccaatttagcgagaaaaatttccaggttaaagactttctggtggaaaatattgatcgcaaatatgacgtgttcattggtaatccgccgtatatcggtcacaaaagcgtggacagcagctacagctacgtgctgcgcaaaatctacggcagcatctaccgcgacaaaggcgatatcagctattgtttctttcagaagagcctgaaatgtctgaaggaaggtggcaaactggtgtttgtgaccagccgctacttctgcgagagctgcagcggtaaagaactgcgtaaattcctgatcgaaaacacgagcatttacaagatcattgatttttacggcatccgcccgttcaaacgcgtgggtatcgatccgatgattatttttctggttcgtacgaagaactggaacaataacattgaaattattcgcccgaacaagattgaaaagaacgaaaagaacaaattcctggatagcctgttcctggacaaaagcgaaaagtgtaaaaagtttagcattagccagaaaagcattaataacgatggctgggttttcgtggacgaagtggagaaaaacattatcgacaaaatcaaagagaaaagcaagttcattctgaaagatatttgccatagctgtcaaggcattatcaccggttgtgatcgcgcctttattgtggaccgtgatatcatcaatagccgtaagatcgaactgcgtctgattaaaccgtggattaaaagcagccatatccgtaagaatgaagttattaagggcgaaaaattcatcatctatagcaacctgattgagaatgaaaccgagtgtccgaatgcgattaaatatatcgaacagtacaagaaacgtctgatggagcgccgcgaatgcaaaaagggcacgcgtaagtggtatgaactgcaatggggccgtaaaccggaaatcttcgaagaaaagaaaattgttttcccgtataaaagctgtgacaatcgttttgcactggataagggtagctattttagcgcagacatttatagcctggttctgaagaaaaatgtgccgttcacctatgagatcctgctgaatatcctgaatagcccgctgtacgagttttactttaagaccttcgcgaaaaagctgggcgagaatctgtacgagtactatccgaacaacctgatgaagctgtgcatcccgagcatcgatttcggcggtgagaacaatattgagaaaaagctgtatgatttctttggtctgacggataaagaaattgagattgtggagaagatcaaagataactgctaagaattcgatatcacccgggaactagtctgcagccctttagtgagggttaattggagtcactaagggttagttagttagattagcagaaagtcaaaagcctccgaccggaggcttttgactaaaacttcccttggggttatcattggggctcactcaaaggcggtaatcagataaaaaaaatccttagctttcgctaaggatgatttctgctagagatggaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgtttactagaggttgatcgggcacgtaagaggttccaactttcaccataatgaaataagatcactaccgggcgtattttttgagttatcgagattttcaggagctaaggaagctaaaatggagaaaaaaatcacgggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaacgctcacccggagtttcgtatggccatgaaagacggtgagctggtgatctgggatagtgttcacccttgttacaccgttttccatgagcaaactgaaacgttttcgtccctctggagtgaataccacgacgatttccggcagtttctccacatatattcgcaagatgtggcgtgttacggtgaaaacctggcctatttccctaaagggtttattgagaatatgttttttgtctcagccaatccctgggtgagtttcaccagttttgatttaaacgtggccaatatggacaacttcttcgcccccgttttcacgatgggcaaatattatacgcaaggcgacaaggtgctgatgccgctggcgatccaggttcatcatgccgtttgtgatggcttccatgtcggccgcatgcttaatgaattacaacagtactgtgatgagtggcagggcggggcgtaataatactagctccggcaaaaaaacgggcaaggtgtcaccaccctgccctttttctttaaaaccgaaaagattacttcgc

SEQ ID NO: 20 - DNA, 18 bp, Artificial sequence
  Synthetic primerr
tttgtaattaagaaggag

SEQ ID NO: 21 - DNA, 18 bp, Artificial sequence
  Synthetic primer
gtagaatccttcttcaac

SEQ ID NO: 22 - DNA, 37 bp, Artificial sequence
  Synthetic primer
ccgaattcgtcgacaacagagtttgatcctggctcag

SEQ ID NO: 23 - DNA, 37 bp, Artificial sequence
  Synthetic primer
cccgggatccaagcttacggctaccttgttacgactt

SEQ ID NO: 24 - DNA, 498 bp, c. autoethanogenum
gagcggccgcaatatgatatttatgtccattgtgaaagggattatattcaactattattccagttacgttcatagaaattttcctttctaaaatattttattccatgtcaagaactctgtttatttcattaaagaactataagtacaaagtataaggcatttgaaaaaataggctagtatattgattgattatttattttaaaatgcctaagtgaaatatatacatattataacaataaaataagtattagtgtaggatttttaaatagagtatctattttcagattaaatttttgattatttgatttacattatataatattgagtaaagtattgactagcaaaattttttgatactttaatttgtgaaatttcttatcaaaagttatatttttgaataatttttattgaaaaatacaactaaaaaggattatagtataagtgtgtgtaattttgtgttaaatttaaagggaggaaatgaacatgaaacatatggaa

SEQ ID NO: 25 - DNA, 563 bp, c. autoethanogenum
ggccgcagatagtcataatagttccagaatagttcaatttagaaattagactaaacttcaaaatgtttgttaaatatataccaaactagtatagatattttttaaatactggacttaaacagtagtaatttgcctaaaaaattttttcaattttttttaaaaaatccttttcaagttgtacattgttatggtaatatgtaattgaagaagttatgtagtaatattgtaaacgtttcttgatttttttacatccatgtagtgcttaaaaaaccaaaatatgtcacatgcaattgtatatttcaaataacaatatttattttctcgttaaattcacaaataatttattaataatatcaataaccaagattatacttaaatggatgtttattttttaacacttttatagtaaatatatttattttatgtagtaaaaaggttataattataattgtatttattacaattaattaaaataaaaaatagggttttaggtaaaattaagttattttaagaagtaattacaataaaaattgaagttatttctttaaggagggaattattca

SEQ ID NO: 26 - DNA, 120 bp, c. autoethanogenum
acagataaaaaaaatatataatacagaagaaaaaattataaatttgtggtataatataaagtatagtaatttaagtttaaaactcgtgaaaacgctaacaaataataggaggtgtattat

SEQ ID NO: 27 - DNA, 350 bp, c. autoethanogenum
acagacaatatagtaatatatgatgttaaaatatcaatatatggttaaaaatctgtatattttttcccattttaattatttgtactataatattacactgagtgtattgtatatttaaaaaatatttggtacaattagttagttaaataaattctaaattgtaaattatcagaatccttattaaggaaatacatagatttaaggagaaatcataaaaaggtgtaatataaactggctaaaattgagcaaaaattgagcaattaagactttttgattgtatctttttatatatttaaggtatataatcttatttatattgggggaacttgatgaataaacatattctagac

SEQ ID NO: 28 - DNA, 1806 bp, Artificial sequence
  synthetic gene
atgtttccgtgcaatgcctatatcgaatatggtgataaaaatatgaacagctttatcgaagatgtggaacagatctacaacttcattaaaaagaacattgatgtggaagaaaagatgcatttcattgaaacctataaacagaaaagcaacatgaagaaagagattagctttagcgaagaatactataaacagaagattatgaacggcaaaaatggcgttgtgtacaccccgccggaaatggcggcctttatggttaaaaatctgatcaacgttaacgatgttattggcaatccgtttattaaaatcattgacccgagctgcggtagcggcaatctgatttgcaaatgttttctgtatctgaatcgcatctttattaagaacattgaggtgattaacagcaaaaataacctgaatctgaaactggaagacatcagctaccacatcgttcgcaacaatctgtttggcttcgatattgacgaaaccgcgatcaaagtgctgaaaattgatctgtttctgatcagcaaccaatttagcgagaaaaatttccaggttaaagactttctggtggaaaatattgatcgcaaatatgacgtgttcattggtaatccgccgtatatcggtcacaaaagcgtggacagcagctacagctacgtgctgcgcaaaatctacggcagcatctaccgcgacaaaggcgatatcagctattgtttctttcagaagagcctgaaatgtctgaaggaaggtggcaaactggtgtttgtgaccagccgctacttctgcgagagctgcagcggtaaagaactgcgtaaattcctgatcgaaaacacgagcatttacaagatcattgatttttacggcatccgcccgttcaaacgcgtgggtatcgatccgatgattatttttctggttcgtacgaagaactggaacaataacattgaaattattcgcccgaacaagattgaaaagaacgaaaagaacaaattcctggatagcctgttcctggacaaaagcgaaaagtgtaaaaagtttagcattagccagaaaagcattaataacgatggctgggttttcgtggacgaagtggagaaaaacattatcgacaaaatcaaagagaaaagcaagttcattctgaaagatatttgccatagctgtcaaggcattatcaccggttgtgatcgcgcctttattgtggaccgtgatatcatcaatagccgtaagatcgaactgcgtctgattaaaccgtggattaaaagcagccatatccgtaagaatgaagttattaagggcgaaaaattcatcatctatagcaacctgattgagaatgaaaccgagtgtccgaatgcgattaaatatatcgaacagtacaagaaacgtctgatggagcgccgcgaatgcaaaaagggcacgcgtaagtggtatgaactgcaatggggccgtaaaccggaaatcttcgaagaaaagaaaattgttttcccgtataaaagctgtgacaatcgttttgcactggataagggtagctattttagcgcagacatttatagcctggttctgaagaaaaatgtgccgttcacctatgagatcctgctgaatatcctgaatagcccgctgtacgagttttactttaagaccttcgcgaaaaagctgggcgagaatctgtacgagtactatccgaacaacctgatgaagctgtgcatcccgagcatcgatttcggcggtgagaacaatattgagaaaaagctgtatgatttctttggtctgacggataaagaaattgagattgtggagaagatcaaagataactgctaa

(19) United States
(12) Patent Application Publication
(10) Pub. No.: US 2011/0256600 A1
(43) Pub. Date: Oct. 20, 2011

(54) Recombinant Microorganisms and Methods of Use Thereof

(75) Inventors: Sean Dennis Simpson, Parnell (NZ); Michael Koepke, Parnell (NZ); FungMin Liew, Parnell (NZ)

(73) Assignee: LanzaTech New Zealand Limited, Parnell (NZ)

(21) Appl. No.: 13/073,069

(22) Filed: Mar. 28, 2011

Related U.S. Application Data
(60) Provisional application No. 61/438,805, filed on Feb. 2, 2011.

(51) Int. Cl. C12P 7/06; C12N 1/00; C12N 1/21

(52) U.S. Cl. 435/161; 435/252.3; 435/243

(57) ABSTRACT

The invention provides a recombinant microorganism capable of producing one or more products by fermentation of a substrate comprising CO, wherein the microorganisms has an increased tolerance to ethanol. The invention also provides, inter alia, methods for the production of ethanol and one or more other products from a substrate comprising CO.

### FIELD OF DISCLOSURE

The present invention relates to methods for the production of biofuels by microbial fermentation and genetically modified micro-organisms with increased tolerance to ethanol.

### BACKGROUND

The growth of most bacteria is affected by relatively low concentrations of alcohols or solvents such as ethanol or butanol. However, the biotechnological production of alcohols is of great interest, for example for use as biofuels. The low natural tolerance of bacteria towards alcohols sets a physical limit for alcohol production, if the alcohol is not removed continuously. The removal of alcohol on the other hand gets far more energy intense and expensive the lower the alcohol concentration (beer strength) (Madson P W: Ethanol distillation: the fundamentals. In: Jaques K A, Lyons T P, Kelsall DR (Eds.): The Alcohol Textbook. 4^{th} edition. 2003, Nottingham University Press: 319-336).

Thus the high toxicity of ethanol and butanol for microorganisms is one of the major problems in bacterial ethanol fermentations as well as the ABE (acetone-butanol-ethanol) fermentation. Only few bacteria, such as some Zymomonas mobilis or Lactococcus strains can tolerate more than 10% ethanol, while the majority of bacteria can only tolerate a maximum of 4-7% ethanol. Butanol is even more toxic for bacterial cells, hardly exceeding levels greater than 1.5-2.5% butanol, while mixtures of different alcohols were shown to act in a synergistic way. Two species of the biotechnologically important genus Clostridium analyzed for alcohol tolerance were shown to tolerate only moderate levels of up to 4-5% or 40-50 g/l ethanol (Rani K S, Seenayya G: High ethanol tolerance of new isolates of Clostridium thermocellum strains SS21 and SS22. World J Microbiol Biotechnol 1999, 2: 173-178; Baskaran S, Ahn H J, Lynd L R: Investigation of the Ethanol

Tolerance of Clostridium thermosaccharolyticum in Continuous Culture. Biotechnol Prog 1995, 3: 276-281) or around 1.5% butanol (Liu S, Qureshi N: How microbes tolerate ethanol and butanol. New Biotechnol 2009, 3-4: 117-121). However, most natural isolates of bacteria shown to have high alcohol tolerance aren't suited as production strains, as they only produce low alcohol yields, or even live on alcohols as carbon source. Thus, there is a need to improve current production strains for higher alcohol tolerance.

Increased butanol levels have been shown to elicit a response similar to a heat shock. Several heat shock stress proteins/chaperons such as ClpB, ClpC, ClpP, DnaK, DnaJ, GreA, GroES, GroEL, GrpE, Hsp18, Hsp90, HtrA, Map, TufA, TufB, or YacI were found to upregulated, both on genetic (Alsaker K V, Paredes C, Papoutsakis E T: Metabolite stress and tolerance in the production of biofuels and chemicals: gene-expression-based systems analysis of butanol, butyrate, and acetate stresses in the anaerobe Clostridium acetobutylicum. Biotechnol Bioeng 2010, 105: 1131-1147; Tomas C A, Beamish J, Papoutsakis E T: Transcriptional Analysis of Butanol Stress and Tolerance in Clostridium acetobutylicum. J Bacteriol 2004, 186: 2006-2018) and protein (Mao S, Luo Y M, Zhang T, Li J, Bao G, Zhu Y, Chen Z, Zhang Y, Li Y, Ma Y: A proteome reference map and comparative proteomic analysis between a wild type Clostridium acetobutylicum DSM 1731 and its mutant with enhanced butanol tolerance and butanol yield. J Proteome Res 2010, 9: 3046-3061) level. Overproduction of Heat shock protein/chaperonin complex GroESL in Clostridium acetobutylicum resulted in a strain which was up to 85% less inhibited by butanol challenge, prolonged metabolism and higher solvent yield compared to the wild-type (Tomas C A, Welker N E, Papoutsakis ET: Overexpression of groESL in Clostridium acetobutylicum results in increased solvent production and tolerance, prolonged metabolism, and changes in the cell's transcriptional program. Appl Environ Microbiol 2003, 69: 4951-49650). The effect of groESL overexpression on ethanol tolerance has not been reported.

It is an object of the invention to overcome one or more of the disadvantages of the prior art, or to at least to provide the public with a useful choice.

### SUMMARY

In a first aspect, the invention provides a recombinant microorganism capable of producing one or more products by fermentation of a substrate comprising CO, wherein the microorganisms has an increased tolerance to ethanol.

In one embodiment, the microorganism is tolerant of ethanol concentrations of at least approximately 5.5% by weight of fermentation broth (ie 55g ethanol/L of fermentation broth). In one particular embodiment, the microorganism is tolerant of ethanol concentrations of at least approximately 6% by weight of fermentation broth.

Preferably, the microorganism is adapted to express, and in one particular embodiment over-express, one or more enzymes adapted to increase tolerance to ethanol.

In one embodiment the one or more enzymes are chosen from the group consisting of stress proteins and chaperones.

In one embodiment, the one or more enzymes are chosen from the group consisting: protein disaggregation chaperone (ClpB), class III stress response-related ATPase (ClpC), ATP-dependent serine protease (ClpP), Hsp70 chaperon (DnaK), Hsp40 chaperon (DnaJ), transcription elongation factor (GreA), Cpn10 chaperonin (GroES), Cpn60 chaperonin (GroEL), heat shock protein (GrpE), heat shock protein (Hsp18), heat shock protein (Hsp90), membrane bound serine protease (HtrA), methionine aminopeptidase (Map), protein chain elongation factor (TufA), protein chain elongation factor (TufB), or Arginine kinase related enzyme (YacI).

In one embodiment, the one or more enzymes are GroES and GroEL.

In one embodiment, the microorganism comprises one or more exogenous nucleic acids adapted to increase expression of one or more nucleic acids native to the microorganism and which encode one or more enzymes referred to herein before. In one embodiment, the one or more exogenous nucleic acid adapted to increase expression is a promoter. In one embodiment, the promoter is a constitutive promoter. In one particular embodiment, the exogenous promoter is a pyruvate:ferredoxin oxidoreductase promoter. In one particular embodiment, the promoter has the nucleic acid sequence of SEQ_ID NO. 5 or a functionally equivalent variant thereof.

In one embodiment, the microorganism comprises one or more exogenous nucleic acids encoding and adapted to express the one or more enzymes referred to herein before.

Preferably, the microorganism comprises one or more exogenous nucleic acids encoding each of GroES (SEQ ID No. 1) and GroEL (SEQ_ID NO. 2). In one particular embodiment nucleic acids encoding each of GroES and GroEL are defined by SEQ_ID NO. 3 and 4 or a functionally equivalent variant thereof.

In one embodiment, the microorganism comprises a nucleic acid construct or vector encoding the one or more enzymes referred to hereinbefore. In one particular embodiment, the construct/vector encodes one or both, and preferably both, of GroES and GroEL.

In one embodiment, nucleic acid construct/vector further comprises an exogenous promoter. In one particular embodiment, the exogenous promoter is a pyruvate:ferredoxin oxidoreductase promoter. In one particular embodiment, the promoter has the nucleic acid sequence of SEQ_ID NO. 5 or a functionally equivalent variant thereof.

In one embodiment, the microorganism is selected from the group of acetogenic bacteria. In certain embodiments the microorganism is selected from the group comprising Clostridium autoethanogenum, Clostridium Ijungdahlii, Clostridium ragsdalei, Clostridium carboxidivorans, Clostridium drakei, Clostridium scatalogenes, Butyribacterium limosum, Acetobacterium woodii, Blautia producta, Eubacterium limosum, Moorella thermoacetica, Moorella thermautotrophica, Oxobacter pfennigii, and Thermoanaerobacter kiuvi.

In one particular embodiment, the microorganism is Clostridium autoethanogenum DSM23693.

In a second embodiment, the invention provides a nucleic acid encoding one or more enzymes, preferably two or more enzymes, which when expressed in a microorganism result in the microorganism having an increased tolerance to ethanol. In one embodiment the enzyme is chosen from the group consisting of stress proteins and chaperones.

In one particular embodiment, the nucleic acid encodes one or more enzyme chosen from the group consisting of ClpB, ClpC, ClpP, DnaK, DnaJ, GreA, GroES, GroEL, GrpE, Hsp18, Hsp90, HtrA, Map, TufA, TufB, or YacI, or functionally equivalent variants thereof, in any order.

In one embodiment, the nucleic acid encodes both GroES and GroEL. In one particular embodiment, the nucleic acid comprises SEQ_ID No 3 and 4, or functionally equivalent variants thereof, in any order. In one embodiment, the nucleic acid comprises SEQ ID_NO. 12, or a functionally equivalent variant thereof.

Preferably, the nucleic acids of this aspect of the invention further comprise a promoter. Preferably, the promoter is a pyruvate:ferredoxin oxidoreductase promoter. In one particular embodiment, the promoter has the nucleic acid sequence of SEQ_ID NO. 5 or a functionally equivalent variant thereof.

In another aspect, the invention provides a nucleic acid construct or vector comprising a nucleic acid of the second aspect of the invention.

In another aspect, the invention provides a nucleic acid consisting of the sequence of any one of SEQ ID NO.s 6, 7, 8, 9, 10, and 11.

In a third aspect, the invention provides an expression construct or vector comprising a nucleic acid sequence encoding one or more enzymes, preferably two or more enzymes, wherein the construct/vector, when expressed in a microorganism, results in the microorganism having an increased tolerance to ethanol.

Preferably, the enzymes are chosen from the group consisting of stress proteins and chaperones.

In one embodiment, the construct/vector comprises a nucleic acid sequence encoding two or more of the enzymes chosen from the group consisting ClpB, ClpC, ClpP, DnaK, DnaJ, GreA, GroES, GroEL, GrpE, Hsp18, Hsp90, HtrA, Map, TufA, TufB, or YacI, in any order.

Preferably, the construct/vector comprises nucleic acid sequences encoding each of GroES (SEQ ID No. 1) and GroEL (SEQ_ID NO. 2). In one particular embodiment, the construct/vector comprises the nucleic acid sequences SEQ_ID NO. 3 and 4 or a functionally equivalent variant thereof, in any order. In one embodiment, the construct/vector comprises SEQ ID_NO. 12, or a functionally equivalent variant thereof.

Preferably, the expression construct/vector further comprises a promoter. Preferably the promoter is a pyruvate:ferredoxin oxidoreductase promoter. In one particular embodiment, the promoter has the nucleic acid sequence of SEQ_ID NO. 5 or a functionally equivalent variant thereof.

In one particular embodiment, the expression construct/vector is a plasmid. In one embodiment, the expression plasmid has the nucleotide sequence SEQ ID No. 17.

In another aspect, the invention provides a host cell comprising one or more nucleic acids of the invention.

In a fourth aspect, the invention provides a composition comprising an expression construct/vector as referred to in the third aspect of the invention and a methylation construct/vector.

Preferably, the composition is able to produce a recombinant microorganism which has increased ethanol tolerance.

In one particular embodiment, the expression construct/vector and/or the methylation construct/vector are plasmids.

In a fifth aspect, the invention provides a method of producing a recombinant microorganism having increased tolerance to ethanol comprising:
a. introduction into a shuttle microorganism of (i) an expression construct/vector of the third aspect of the invention and (ii) a methylation construct/vector comprising a methyltransferase gene;
b. expression of the methyltransferase gene;
c. isolation of one or more constructs/vectors from the shuttle microorganism; and,
d. introduction of at least the expression construct/vector into a destination microorganism.

In one embodiment, the methyltransferase gene of step B is expressed consitutively. In another embodiment, expression of the methyltransferase gene of step B is induced.

In one embodiment, both the methylation construct/vector and the expression construct/vector are isolated in step C. In another embodiment, only the expression construct/vector is isolated in step C.

In one embodiment, only the expression construct/vector is introduced into the destination microorganism. In another embodiment, both the expression construct/vector and the methylation construct/vector are introduced into the destination microorganism.

In a related aspect, the invention provides a method of producing a recombinant microorganism having increased tolerance to ethanol comprising:
a. methylation of an expression construct/vector of the third aspect of the invention in vitro by a methyltransferase;
b. introduction of the expression construct/vector into a destination microorganism.

In a further related aspect, the invention provides a method of producing a recombinant microorganism having increased tolerance to ethanol comprising:
a. introduction into the genome of a shuttle microorganism of a methyltransferase gene
b. introduction of an expression construct/vector of the third aspect of the invention into the shuttle microorganism
c. isolation of one or more constructs/vectors from the shuttle microorganism; and,
d. introduction of at least the expression construct/vector into a destination microorganism.

In a sixth aspect, the invention provides a method for the production of ethanol and/or one or more other products by microbial fermentation comprising fermenting a substrate comprising CO using a recombinant microorganism of the first aspect of the invention.

The invention also provides a method for reducing the total atmospheric carbon emissions from an industrial process.

In one embodiment the method comprises the steps of:
(a) providing a substrate comprising CO to a bioreactor containing a culture of one or more microorganism of the first aspect of the invention; and
(b) anaerobically fermenting the culture in the bioreactor to produce one or more products including ethanol.

In another embodiment the method comprises the steps of:
(a) capturing CO-containing gas produced as a result of the industrial process, before the gas is released into the atmosphere;
(b) the anaerobic fermentation of the CO-containing gas to produce one or more products including ethanol by a culture containing one or more microorganism of the first aspect of the invention.

In one embodiment, the ethanol concentration in the fermentation broth is at least approximately 5.5% by weight. In another embodiment, the ethanol concentration in the fermentation broth is at least approximately 6% by weight.

In particular embodiments of the method aspects, the microorganism is maintained in an aqueous culture medium.

In particular embodiments of the method aspects, the fermentation of the substrate takes place in a bioreactor.

Preferably, the substrate comprising CO is a gaseous substrate comprising CO. In one embodiment, the substrate comprises an industrial waste gas. In certain embodiments, the gas is steel mill waste gas or syngas.

In one embodiment, the substrate will typically contain a major proportion of CO, such as at least about 20% to about 100% CO by volume, from 20% to 70% CO by volume, from 30% to 60% CO by volume, and from 40% to 55% CO by volume. In particular embodiments, the substrate comprises about 25%, or about 30%, or about 35%, or about 40%, or about 45%, or about 50% CO, or about 55% CO, or about 60% CO by volume.

While it is not necessary for the substrate to contain any hydrogen, the presence of H₂ should not be detrimental to product formation in accordance with methods of the invention. In particular embodiments, the presence of hydrogen results in an improved overall efficiency of alcohol production. For example, in particular embodiments, the substrate may comprise an approx 2:1, or 1:1, or 1:2 ratio of H₂:CO. In one embodiment the substrate comprises about 30% or less H₂ by volume, 20% or less H₂ by volume, about 15% or less H₂ by volume or about 10% or less H₂ by volume. In other embodiments, the substrate stream comprises low concentrations of H₂, for example, less than 5%, or less than 4%, or less than 3%, or less than 2%, or less than 1%, or is substantially hydrogen free. The substrate may also contain some CO₂ for example, such as about 1% to about 80% CO₂ by volume, or 1% to about 30% CO₂ by volume.

In certain embodiments the methods further comprise the step of recovering the one or more products from the fermentation broth, the fermentation broth.

In a seventh aspect, the invention provides ethanol and/or one or more other product when produced by the method of the sixth aspect.

The invention may also be said broadly to consist in the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

### BRIEF DESCRIPTION OF THE FIGURES

These and other aspects of the present invention, which should be considered in all its novel aspects, will become apparent from the following description, which is given by way of example only, with reference to the accompanying figures, in which:

FIG. 1 shows Ethanol tolerance of Clostridium autoethanogenum DSM23693 in serum bottles.

FIG. 2 shows Expression of the pyruvate:ferredoxin oxidoreductase during a normal batch fermentation run compared to over 200 genes of interest.

FIG. 3 illustrates the DNA sequencing of groESL insert in plasmid pCR-Blunt-GroESL.

FIG. 4 shows a map of the plasmid pMTL85246-GroESL.

FIG. 5 illustrates the DNA sequencing alignment of P_{pfor} and groESL insert in plasmid pMTL85246-GroESL.

FIG. 6 shows a methylation plasmid.

FIG. 7 shows detection of ermB (400 bp) and groESL (2 kbp) from PCR of plasmid isolated from transformed C. autoethanogenum DSM23693. Ladder=1 KB Plus DNA ladder (Invitrogen); 132 ermB from non-template control; 2=ermB from plasmid isolated from C. autoethanogenum; 332 ermB from original plasmid pMTL 85246-GroESL (as positive control); 4=groESL from non-template control; 5=groESL from plasmid isolated from C. autoethanogenum; 6=groESL from original plasmid pMTL 85246-GroESL (as positive control).

FIG. 8 illustrates an ethanol challenge experiment with C. autoethanogenum DSM23693 wild-type (WT) and transformed strain carrying plasmid pMTL 85246-GroESL.

FIG. 9 SEQ_ID NO. 1: Amino acid sequence of Heat shock protein/chaperonin GroES from C. autoethanogenum.

FIG. 10 SEQ_ID NO. 2: Amino acid sequence of Heat shock protein/chaperonin GroEL from C. autoethanogenum.

FIG. 11 SEQ_ID NO. 3: Nucleic acid sequence of Heat shock protein/chaperonin gene groES from C. autoethanogenum.

FIG. 12 SEQ_ID NO. 4: Nucleic acid sequence of Heat shock protein/chaperonin gene groES from C. autoethanogenum.

FIG. 13 SEQ_ID NO. 5: Nucleic acid sequence of Pyruvate:ferredoxin promoter P_{PFOR} from C. autoethanogenum.

FIG. 14 SEQ_ID NO. 12: Nucleic acid sequence of SOE PCR product from mutated groESL operon of C. autoethanogenum.

FIG. 15 SEQ_ID NO. 13: Nucleic acid sequence of oligonulceotide M13 Forward (−20).

FIG. 16 SEQ_ID NO. 14: Nucleic acid sequence of oligonulceotide M13 Reverse.

FIG. 17 Seq. ID 15: Nucleic acid sequence of E. coli-Clostridium shuttle vector pMTL85141.

FIG. 18 Seq. ID 16: Nucleic acid sequence of E. coli-Clostridium shuttle vector pMTL82254.

FIG. 19 SEQ_ID NO. 17: Nucleic acid sequence of groESL overexpression plasmid pMTL85246-GroESL.

FIG. 20 SEQ_ID NO. 18: Amino acid sequence of designed Type II methyltransferase.

FIG. 21 SEQ_ID NO. 19: Nucleic acid sequence of methylation plasmid.

FIG. 22 SEQ_ID NO. 20: Nucleic acid sequence of oligonucleotide ermB-F.

FIG. 23 SEQ_ID NO. 21: Nucleic acid sequence of oligonucleotide ermB-R.

FIG. 24 SEQ_ID NO. 22: Nucleic acid sequence of oligonucleotide fD1.

FIG. 25 SEQ_ID NO. 23: Nucleic acid sequence of oligonucleotide rP2.

FIG. 26: SEQ_ID No. 24: Nucleic acid sequence of Clostridium autoethanogenum phosphotransacetylase/acetate kinase promoter region

FIG. 27: SEQ_ID No. 25: Nucleic acid sequence of Clostridium autoethanogenum Wood-Ljungdahl cluster promoter region

FIG. 28: SEQ_ID No. 26: Nucleic acid sequence of Clostridium autoethanogenum RnF operon promoter region

FIG. 29: SEQ_ID No. 27: Nucleic acid sequence of Clostridium autoethanogenum ATP synthase operon promoter region

FIG. 30: Table of exemplary information for enzymes of use in the invention. The protein accession number is followed by the gene ID (GenBank) for each microorganism listed.

FIG. 31: SEQ_ID NO. 28: Nucleic acid sequence of designed Type II methyltransferase gene.

### DETAILED DESCRIPTION OF THE INVENTION

The following is a description of the present invention, including preferred embodiments thereof, given in general terms. The invention is further elucidated from the disclosure given under the heading “Examples” herein below, which provides experimental data supporting the invention, specific examples of various aspects of the invention, and means of performing the invention.

The invention provides a recombinant microorganism capable of producing ethanol or, ethanol and one or more other products, by fermentation of a substrate comprising CO, wherein the microorganisms has an increased tolerance to ethanol.

Solvents and alcohols are often toxic to microorganisms, even at very low concentrations. This can increase costs and limit the commercial viability of methods for the production of alcohols and other products by bacterial fermentation. The inventors have developed recombinant microorganisms which surprisingly have increased ethanol tolerance and thus may be used to improve efficiencies of the production of ethanol and/or other products by fermentation of substrates comprising CO.

### Definitions

As referred to herein, a “fermentation broth” is a culture medium comprising at least a nutrient media and bacterial cells.

As referred to herein, a shuttle microorganism is a microorganism in which a methyltransferase enzyme is expressed and is distinct from the destination microorganism.

As referred to herein, a destination microorganism is a microorganism in which the genes included on an expression construct/vector are expressed and is distinct from the shuttle microorganism.

The term “main fermentation product” is intended to mean the one fermentation product which is produced in the highest concentration and/or yield.

The terms “increasing the efficiency”, “increased efficiency” and the like, when used in relation to a fermentation process, include, but are not limited to, increasing one or more of the rate of growth of microorganisms catalysing the fermentation, the growth and/or product production rate at elevated ethanol concentrations, the volume of desired product (such as alcohols) produced per volume of substrate consumed, the rate of production or level of production of the desired product, and the relative proportion of the desired product produced compared with other by-products of the fermentation.

“Increased tolerance to ethanol” and like terms should be taken to mean that the recombinant microorganism has a higher tolerance to ethanol as compared to a parental microorganism. Tolerance may be measured in terms of the survival of a microorganism or population of microorganisms, the growth rate of a microorganism or population of microorganisms and/or the rate of production of one or more products by a microorganism or population of microorganisms in the presence of ethanol. In one particular embodiment of the invention, it is measured in terms of the ability of a microorganism or population of microorganisms to grow in the presence of ethanol concentrations which are typically toxic to the parental microorganism.

The phrase “substrate comprising carbon monoxide” and like terms should be understood to include any substrate in which carbon monoxide is available to one or more strains of bacteria for growth and/or fermentation, for example.

The phrase “gaseous substrate comprising carbon monoxide” and like phrases and terms includes any gas which contains a level of carbon monoxide. In certain embodiments the substrate contains at least about 20% to about 100% CO by volume, from 20% to 70% CO by volume, from 30% to 60% CO by volume, and from 40% to 55% CO by volume. In particular embodiments, the substrate comprises about 25%, or about 30%, or about 35%, or about 40%, or about 45%, or about 50% CO, or about 55% CO, or about 60% CO by volume.

While it is not necessary for the substrate to contain any hydrogen, the presence of H₂ should not be detrimental to product formation in accordance with methods of the invention. In particular embodiments, the presence of hydrogen results in an improved overall efficiency of alcohol production. For example, in particular embodiments, the substrate may comprise an approx 2:1, or 1:1, or 1:2 ratio of H2:CO. In one embodiment the substrate comprises about 30% or less H₂ by volume, 20% or less H₂ by volume, about 15% or less H₂ by volume or about 10% or less H₂ by volume. In other embodiments, the substrate stream comprises low concentrations of H2, for example, less than 5%, or less than 4%, or less than 3%, or less than 2%, or less than 1%, or is substantially hydrogen free. The substrate may also contain some CO₂ for example, such as about 1% to about 80% CO₂ by volume, or 1% to about 30% CO₂ by volume. In one embodiment the substrate comprises less than or equal to about 20% CO₂ by volume. In particular embodiments the substrate comprises less than or equal to about 15% CO₂ by volume, less than or equal to about 10% CO₂ by volume, less than or equal to about 5% CO₂ by volume or substantially no CO₂.

In the description which follows, embodiments of the invention are described in terms of delivering and fermenting a “gaseous substrate containing CO”. However, it should be appreciated that the gaseous substrate may be provided in alternative forms. For example, the gaseous substrate containing CO may be provided dissolved in a liquid. Essentially, a liquid is saturated with a carbon monoxide containing gas and then that liquid is added to the bioreactor.

This may be achieved using standard methodology. By way of example, a microbubble dispersion generator (Hensirisak et. al. Scale-up of microbubble dispersion generator for aerobic fermentation; Applied Biochemistry and Biotechnology Volume 101, Number 3/October, 2002) could be used. By way of further example, the gaseous substrate containing CO may be adsorbed onto a solid support. Such alternative methods are encompassed by use of the term “substrate containing CO” and the like.

In particular embodiments of the invention, the CO-containing gaseous substrate is an industrial off or waste gas. “Industrial waste or off gases” should be taken broadly to include any gases comprising CO produced by an industrial process and include gases produced as a result of ferrous metal products manufacturing, non-ferrous products manufacturing, petroleum refining processes, gasification of coal, gasification of biomass, electric power production, carbon black production, and coke manufacturing. Further examples may be provided elsewhere herein.

Unless the context requires otherwise, the phrases “fermenting”, “fermentation process” or “fermentation reaction” and the like, as used herein, are intended to encompass both the growth phase and product biosynthesis phase of the process. As will be described further herein, in some embodiments the bioreactor may comprise a first growth reactor and a second fermentation reactor. As such, the addition of metals or compositions to a fermentation reaction should be understood to include addition to either or both of these reactors.

The term “bioreactor” includes a fermentation device consisting of one or more vessels and/or towers or piping arrangement, which includes the Continuous Stirred Tank Reactor (CSTR), Immobilized Cell Reactor (ICR), Trickle Bed Reactor (TBR), Bubble Column, Gas Lift Fermenter, Static Mixer, or other vessel or other device suitable for gas-liquid contact. As is described herein after, in some embodiments the bioreactor may comprise a first growth reactor and a second fermentation reactor. As such, when referring to the addition of substrate to the bioreactor or fermentation reaction it should be understood to include addition to either or both of these reactors where appropriate.

When used in relation to the products of a fermentation in accordance with the invention “one or more other products” is intended to include acetate and 2,3-butanediol, for example. It should be appreciated that the methods of the invention are applicable to methods intended for the production and recovery of products other than ethanol, but where ethanol is produced as a by-product and may have an impact on the efficiency of growth of and production by one or more microorganisms.

The term “acetate” includes both acetate salt alone and a mixture of molecular or free acetic acid and acetate salt, such as the mixture of acetate salt and free acetic acid present in a fermentation broth as described herein. The ratio of molecular acetic acid to acetate in the fermentation broth is dependent upon the pH of the system.

“Exogenous nucleic acids” are nucleic acids which originate outside of the microorganism to which they are introduced. Exogenous nucleic acids may be derived from any appropriate source, including, but not limited to, the microorganism to which they are to be introduced, strains or species of microorganisms which differ from the organism to which they are to be introduced, or they may be artificially or recombinantly created. In one embodiment, the exogenous nucleic acids represent nucleic acid sequences naturally present within the microorganism to which they are to be introduced, and they are introduced to increase expression of or over-express a particular gene (for example, by increasing the copy number of the sequence (for example a gene). In another embodiment, the exogenous nucleic acids represent nucleic acid sequences not naturally present within the microorganism to which they are to be introduced and allow for the expression of a product not naturally present within the microorganism or increased expression of a gene native to the microorganism (for example in the case of introduction of a regulatory element such as a promoter). The exogenous nucleic acid may be adapted to integrate into the genome of the microorganism to which it is to be introduced or to remain in an extra-chromosomal state.

It should be appreciated that the invention may be practised using nucleic acids whose sequence varies from the sequences specifically exemplified herein provided they perform substantially the same function. For nucleic acid sequences that encode a protein or peptide this means that the encoded protein or peptide has substantially the same function. For nucleic acid sequences that represent promoter sequences, the variant sequence will have the ability to promote expression of one or more genes. Such nucleic acids may be referred to herein as “functionally equivalent variants”. By way of example, functionally equivalent variants of a nucleic acid include allelic variants, fragments of a gene, genes which include mutations (deletion, insertion, nucleotide substitutions and the like) and/or polymorphisms and the like. Homologous genes from other microorganisms may also be considered as examples of functionally equivalent variants of the sequences specifically exemplified herein. These include homologous genes in species such as Escherichia coli, Bacillus subtilis, Clostridium acetobutylicum, Clostridium ljungdahlii, Clostridium carboxidivorans could be used, details of which are publicly available on websites such as Genbank or NCBI. The phrase “functionally equivalent variants” should also be taken to include nucleic acids whose sequence varies as a result of codon optimisation for a particular organism.

“Functionally equivalent variants” of a nucleic acid herein will preferably have at least approximately 70%, preferably approximately 80%, more preferably approximately 85%, preferably approximately 90%, preferably approximately 95% or greater nucleic acid sequence identity with the nucleic acid identified.

It should also be appreciated that the invention may be practised using polypeptides whose sequence varies from the amino acid sequences specifically exemplified herein. These variants may be referred to herein as “functionally equivalent variants”. A functionally equivalent variant of a protein or a peptide includes those proteins or peptides that share at least 40%, preferably 50%, preferably 60%, preferably 70%, preferably 75%, preferably 80%, preferably 85%, preferably 90%, preferably 95% or greater amino acid identity with the protein or peptide identified and has substantially the same function as the peptide or protein of interest. Such variants include within their scope fragments of a protein or peptide wherein the fragment comprises a truncated form of the polypeptide wherein deletions may be from 1 to 5, to 10, to 15, to 20, to 25 amino acids, and may extend from residue 1 through 25 at either terminus of the polypeptide, and wherein deletions may be of any length within the region; or may be at an internal location. Functionally equivalent variants of the specific polypeptides herein should also be taken to include polypeptides expressed by homologous genes in other species of bacteria, for example as exemplified in the previous paragraph.

“Substantially the same function” as used herein is intended to mean that the nucleic acid or polypeptide is able to perform the function of the nucleic acid or polypeptide of which it is a variant. For example, a variant of an enzyme of the invention will be able to catalyse the same reaction as that enzyme. However, it should not be taken to mean that the variant has the same level of activity as the polypeptide or nucleic acid of which it is a variant.

One may assess whether a functionally equivalent variant has substantially the same function as the nucleic acid or polypeptide of which it is a variant using any number of known methods. However, by way of example, the methods outlined in Zietkiewicz et al (Hsp70 chaperone machine remodels protein aggregates at the initial step of Hsp70-Hsp100-dependent disaggregation, J Biol Chem 2006, 281: 7022-7029), Zzaman et al (The DnaK-DnaJ-GrpE chaperone system activates inert wild type pi initiator protein of R6K into a form active in replication initiation, J Biol Chem 2004, 279: 50886-50894), Zavilgelsky et al (Role of Hsp70 (DnaK-DnaJ-GrpE) and Hsp100 (ClpA and ClpB) chaperones in refolding and increased thermal stability of bacterial luciferases in Escherichia coli cells, Biochemistry (Mosc) 2002, 67: 986-992), or Konieczny and Liberek (Cooperative action of Escherichia coli ClpB protein and DnaK chaperone in the activation of a replication initiation protein, J Biol Chem 2002, 277: 18483-18488) may be used to assess enzyme activity.

A “stress protein”, as used herein, is intended to include any protein which is expressed in response to stress and includes for example, heat shock proteins, chaperon complexes, transcription elongation factors, proteases, and petidases.

A “chaperone”, as used herein, is intended to include any peptide or protein which is involved in controlling and maintaining the correct folding of proteins and enzymes in their active state, and includes those proteins involved in refolding misfolded and aggregated proteins, for example after exposure to heat or alcohols.

“Over-express”, “over expression” and like terms and phrases when used in relation to the invention should be taken broadly to include any increase in expression of one or more protein as compared to the expression level of the protein of a parental microorganism under the same conditions. It should not be taken to mean that the protein is expressed at any particular level.

A “parental microorganism” is a microorganism used to generate a recombinant microorganism of the invention. The parental microorganism may be one that occurs in nature (ie a wild type microorganism) or one that has been previously modified but which does not express or over-express one or more of the enzymes the subject of the present invention. Accordingly, the recombinant microorganisms of the invention have been modified to express or over-express one or more enzymes that were not expressed or over-expressed in the parental microorganism.

The terms nucleic acid “constructs” or “vectors” and like terms should be taken broadly to include any nucleic acid (including DNA and RNA) suitable for use as a vehicle to transfer genetic material into a cell. The terms should be taken to include plasmids, viruses (including bacteriophage), cosmids and artificial chromosomes. Constructs or vectors may include one or more regulatory elements, an origin of replication, a multicloning site and/or a selectable marker, among other elements, sites and markers. In one particular embodiment, the constructs or vectors are adapted to allow expression of one or more genes encoded by the construct or vector. Nucleic acid constructs or vectors include naked nucleic acids as well as nucleic acids formulated with one or more agents to facilitate delivery to a cell (for example, liposome-conjugated nucleic acid, an organism in which the nucleic acid is contained).

As discussed herein before, the invention provides a recombinant microorganism capable of producing ethanol and one or more other products by fermentation of a substrate comprising CO, wherein the microorganism has an increased tolerance to ethanol.

In one embodiment, the microorganism is tolerant of ethanol concentrations of at least approximately 5.5% by weight of fermentation broth. In one particular embodiment, the microorganism is tolerant of ethanol concentrations of at least approximately 6% by weight of fermentation broth.

In particular embodiments, the microorganism is adapted to express one or more enzyme adapted to increase tolerance to ethanol which are not naturally present in the parental microorganism, or over-express one or more enzyme adapted to increase tolerance to ethanol which are naturally present in the parental microorganism.

The microorganism may be adapted to express or over-express the one or more enzymes by any number of recombinant methods including, for example, increasing expression of native genes within the microorganism (for example, by introducing a stronger or constitutive promoter to drive expression of a gene), increasing the copy number of a gene encoding a particular enzyme by introducing exogenous nucleic acids encoding and adapted to express the enzyme, introducing an exogenous nucleic acid encoding and adapted to express an enzyme not naturally present within the parental microorganism.

In certain embodiments, the parental microorganism may be transformed to provide a combination of increased or over-expression of one or more genes native to the parental microorganism and introduction of one or more genes not native to the parental microorganism.

In one embodiment the one or more enzymes are chosen from the group consisting of stress proteins and chaperones.

In one embodiment, the one or more enzymes are chosen from the group consisting: protein disaggregation chaperone (ClpB), class III stress response-related ATPase (ClpC), ATP-dependent serine protease (ClpP), Hsp70 chaperon (DnaK), Hsp40 chaperon (DnaJ), transcription elongation factor (GreA), Cpn10 chaperonin (GroES), Cpn60 chaperonin (GroEL), heat shock protein (GrpE), heat shock protein (Hsp18), heat shock protein (Hsp90), membrane bound serine protease (HtrA), methionine aminopeptidase (Map), protein chain elongation factor (TufA), protein chain elongation factor (TufB), or Arginine kinase related enzyme (YacI), and functionally equivalent variants of any one thereof.

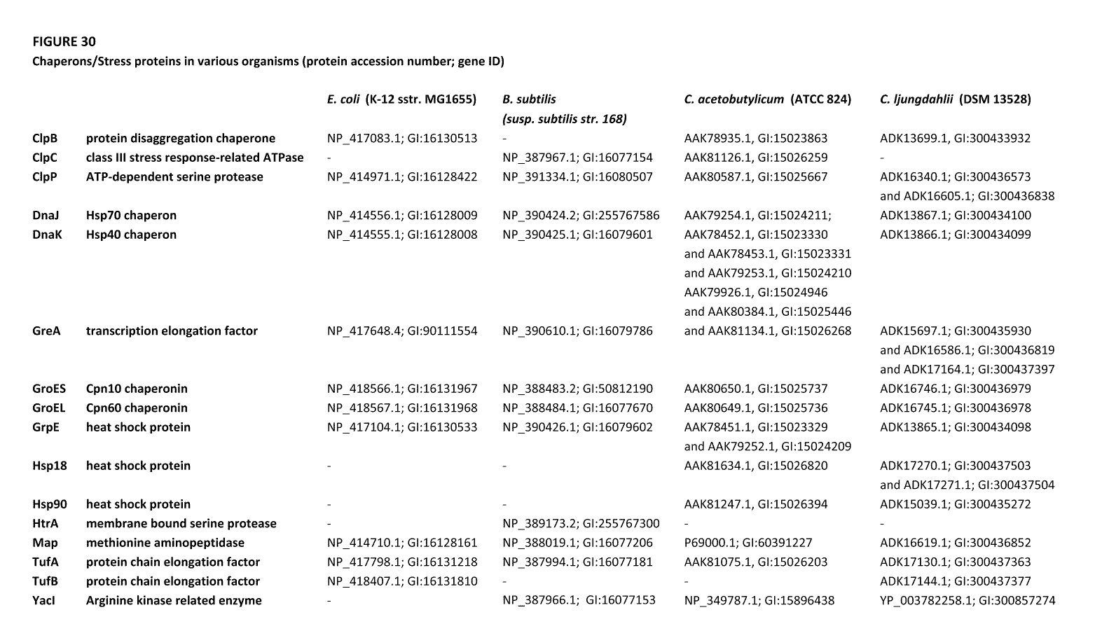

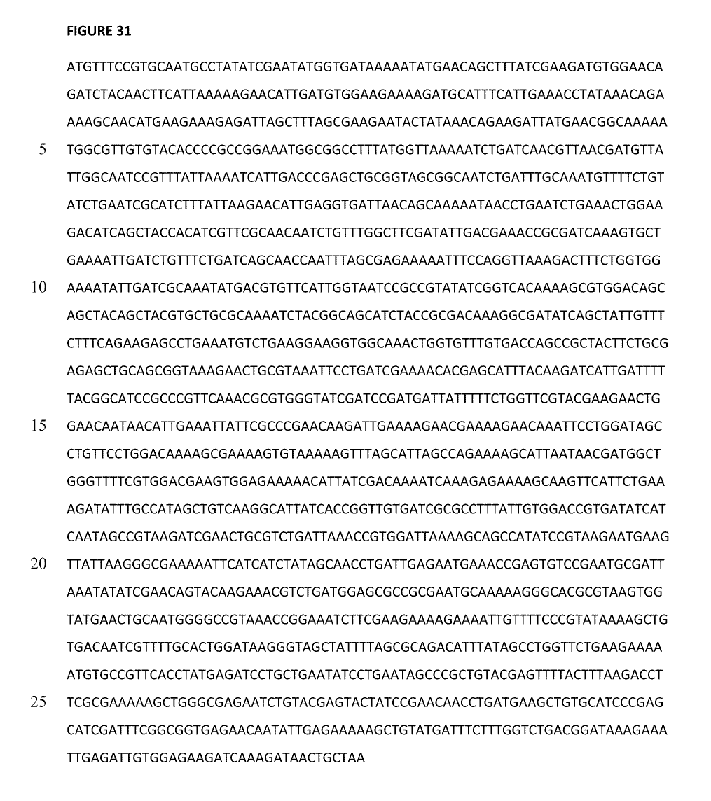

Exemplary nucleic acid and amino acid sequence information for the above enzymes are found in Gen Bank, as outlined in the table in FIG. 30.

In one embodiment, the one or more enzymes are GroES and GroEL.

In one embodiment, the microorganism comprises one or more exogenous nucleic acids adapted to increase expression of one or more nucleic acids native to the microorganism and which one or more nucleic acids encode one or more enzymes referred to herein before. In one embodiment, the one or more exogenous nucleic acid adapted to increase expression is a promoter. In one embodiment, the promoter is a constitutive promoter that is preferably highly active under appropriate fermentation conditions. However, inducible promoters may also be employed. In preferred embodiments, the promoter is selected from the group comprising phosphotransacetylase/acetate kinase operon promoter (SEQ_ID No. 24), pyruvate:ferredoxin oxidoreductase (SEQ_ID No. 5), the Wood-Ljungdahl gene cluster (SEQ_ID No 25), Rnf operon (SEQ_ID No 26) or the ATP synthase operon (SEQ_ID No 27). Preferably, the promoter is a pyruvate:ferredoxin oxidoreductase promoter. In one particular embodiment, the promoter has the nucleic acid sequence of SEQ_ID NO. 5 or a functionally equivalent variant thereof. It will be appreciated by those of skill in the art that other promoters which can direct expression, preferably a high level of expression under appropriate fermentation conditions, would be effective as alternatives to the exemplified embodiments.

In one embodiment, the microorganism comprises one or more exogenous nucleic acids encoding and adapted to express the one or more enzymes referred to herein before. In one embodiment, the microorganisms comprises one or more exogenous nucleic acid encoding and adapted to express at least two enzymes adapted to increase tolerance to ethanol. In other embodiments, the microorganism comprises one or more exogenous nucleic acid encoding and adapted to express at least 3, at least 4, at least 5 or at least 6 enzymes adapted to increase tolerance to ethanol.

In one embodiment, the microorganism comprises one or more exogenous nucleic acid encoding each of GroES and GroEL, or a functionally equivalent variant of either or both. In one particular embodiment nucleic acids encoding each of GroES and GroEL are defined by SEQ_ID NO. 3 and 4 or a functionally equivalent variant thereof. In one embodiment, the microorganism comprises a nucleic acid comprises SEQ ID_NO. 12, or a functionally equivalent variant thereof.

In one embodiment, the microorganism comprises a nucleic acid construct or vector, for example a plasmid, encoding the one or more enzymes referred to hereinbefore. In one particular embodiment, the construct encodes one or both, and preferably both, of GroES and GroEL. In one embodiment, the construct or vector comprises nucleic acid sequences encoding each of GroES (SEQ ID No. 1) and GroEL (SEQ_ID NO. 2). In one particular embodiment, the vector comprises the nucleic acid sequences SEQ_ID NO. 3 and 4 or a functionally equivalent variant thereof, in any order. In one embodiment, the vector/construct comprises SEQ ID_NO. 12, or a functionally equivalent variant thereof.

In one embodiment, the nucleic acid construct/vector further comprises an exogenous promoter adapted to promote expression of the one or more enzymes encoded by the exogenous nucleic acids.

In one embodiment the promoter is a constitutive promoter that is preferably highly active under appropriate fermentation conditions. However, inducible promoters may also be employed. In preferred embodiments, the promoter is selected from the group comprising phosphotransacetylase/acetate kinase operon promoter (SEQ ID NO. 24), pyruvate:ferredoxin oxidoreductase (SEQ_ID No. 5), the Wood-Ljungdahl gene cluster (SEQ_ID No 25), Rnf operon (SEQ_ID No 26) or the ATP synthase operon ((SEQ_ID No 27). Preferably, the promoter is a pyruvate:ferredoxin oxidoreductase promoter. In one particular embodiment, the promoter has the nucleic acid sequence of SEQ_ID NO. 5 or a functionally equivalent variant thereof. It will be appreciated by those of skill in the art that other promoters which can direct expression, preferably a high level of expression under appropriate fermentation conditions, would be effective as alternatives to the exemplified embodiments.

In one embodiment, the exogenous nucleic acid is an expression plasmid having the nucleotide sequence SEQ ID No. 17.

In one embodiment, the nucleic acids encoding the one or more enzymes, and optionally the promoter, are integrated into the genome of the microorganism. In other embodiment, the nucleic acids encoding the one or more enzymes are not integrated into the genome of the microorganism.

In one embodiment, the parental microorganism is selected from the group of acetogenic bacteria. In certain embodiments the microorganism is selected from the group comprising Clostridium autoethanogenum, Clostridium ljungdahlii, Clostridium ragsdalei, Clostridium carboxidivorans, Clostridium drakei, Clostridium scatalogenes, Butyribacterium limosum, Acetobacterium woodii, Blautia producta, Eubacterium limosum, Moorella thermoacetica, Moorella thermautotrophica, Oxobacter pfennigii, and Thermoanaerobacter kiuvi.

In one particular embodiment, the parental microorganism is selected from the cluster of ethanologenic, acetogenic Clostridia comprising the species C. autoethanogenum, C. ljungdahlii, and C. ragsdalei and related isolates. These include but are not limited to strains C. autoethanogenum JAI-1^{T} (DSM10061) [Abrini J, Naveau H, Nyns E-J: Clostridium autoethanogenum, sp. nov., an anaerobic bacterium that produces ethanol from carbon monoxide. Arch Microbiol 1994, 4: 345-351], C. autoethanogenum LBS1560 (DSM19630) [Simpson S D, Forster R L, Tran P T, Rowe M J, Warner I L: Novel bacteria and methods thereof. International patent 2009, WO/2009/064200], C. autoethanogenum LBS1561 (DSM23693), C. ljungdahlii PETC^{T} (DSM13528 =ATCC 55383) [Tanner R S, Miller L M, Yang D: Clostridium ljungdahlii sp. nov., an Acetogenic Species in Clostridial rRNA Homology Group I. Int J Syst Bacteriol 1993, 43: 232-236], C. ljungdahlii ERI-2 (ATCC 55380) [Gaddy J L: Clostridium stain which produces acetic acid from waste gases. 1997, U.S. Pat. No. 5,593,886], C. ljungdahlii C-01 (ATCC 55988) [Gaddy J L, Clausen E C, Ko C-W: Microbial process for the preparation of acetic acid as well as solvent for its extraction from the fermentation broth. 2002, U.S. Pat. No. 6,368,819], C. ljungdahlii O-52 (ATCC 55989) [Gaddy J L, Clausen EC, Ko C-W: Microbial process for the preparation of acetic acid as well as solvent for its extraction from the fermentation broth. 2002, U.S. Pat. No. 6,368,819], C. ragsdalei P11^{T} (ATCC BAA-622) [Huhnke R L, Lewis R S, Tanner R S: Isolation and Characterization of novel Clostridial Species. International patent 2008, WO 2008/028055], related isolates such as “C. coskatii” [Zahn J A, Saxena J, Do Y, Patel M, Fishein S, Datta R, Tobey R: Clostridium coskatii, sp. nov., an Anaerobic Bacterium that Produces Ethanol from Synthesis Gas. Poster SIM Annual Meeting and Exhibition, San Francisco, 2010], or mutated strains such as C. ljungdahlii OTA-1 (Tirado-Acevedo O. Production of Bioethanol from Synthesis Gas Using Clostridium ljungdahlii. PhD thesis, North Carolina State University, 2010). These strains form a subcluster within the Clostridial rRNA cluster I, and their 16S rRNA gene is more than 99% identical with a similar low GC content of around 30%. However, DNA-DNA reassociation and DNA fingerprinting experiments showed that these strains belong to distinct species [Huhnke R L, Lewis R S, Tanner R S: Isolation and Characterization of novel Clostridial Species. International patent 2008, WO 2008/028055].

All species of this cluster have a similar morphology and size (logarithmic growing cells are between 0.5-0.7×3-5 μm), are mesophilic (optimal growth temperature between 30-37° C.) and strictly anaerobe [Tanner R S, Miller L M, Yang D: Clostridium ljungdahlii sp. nov., an Acetogenic Species in Clostridial rRNA Homology Group I. Int J Syst Bacteriol 1993, 43: 232-236; Abrini J, Naveau H, Nyns E-J: Clostridium autoethanogenum, sp. nov., an anaerobic bacterium that produces ethanol from carbon monoxide. Arch Microbiol 1994, 4: 345-351; Huhnke R L, Lewis R S, Tanner R S: Isolation and Characterization of novel Clostridial Species. International patent 2008, WO 2008/028055]. Moreover, they all share the same major phylogenetic traits, such as same pH range (pH 4-7.5, with an optimal initial pH of 5.5-6), strong autotrophic growth on CO containing gases with similar growth rates, and a similar metabolic profile with ethanol and acetic acid as main fermentation end product, and small amounts of 2,3-butanediol and lactic acid formed under certain conditions. [Tanner R S, Miller L M, Yang D: Clostridium ljungdahlii sp. nov., an Acetogenic Species in Clostridial rRNA Homology Group I. Int J Syst Bacteriol 1993, 43: 232-236; Abrini J, Naveau H, Nyns E-J: Clostridium autoethanogenum, sp. nov., an anaerobic bacterium that produces ethanol from carbon monoxide. Arch Microbiol 1994, 4: 345-351; Huhnke R L, Lewis R S, Tanner R S: Isolation and Characterization of novel Clostridial Species. International patent 2008, WO 2008/028055]. Indole production was observed with all three species as well. However, the species differentiate in substrate utilization of various sugars (e.g. rhamnose, arabinose), acids (e.g. gluconate, citrate), amino acids (e.g. arginine, histidine), or other substrates (e.g. betaine, butanol). Moreover some of the species were found to be auxotroph to certain vitamins (e.g. thiamine, biotin) while others were not.

In one particular embodiment, the parental microorganism is Clostridium autoethanogenum DSM23693.

In one embodiment, the parental microorganism lacks one or more genes encoding the enzymes referred to herein before.

The invention also provides nucleic acids and nucleic acid constructs of use in generating a recombinant microorganism of the invention.

The nucleic acids may encode one or more enzymes, which when expressed in a microorganism, result in the microorganism having an increased tolerance to ethanol. In one particular embodiment, the invention provides a nucleic acid encoding two or more enzymes, which when expressed in a microorganism, results in the microorganism having an increased tolerance to ethanol. In one particular embodiment, the two or more enzymes are chosen from ClpB, ClpC, ClpP, DnaK, DnaJ, GreA, GroES, GroEL, GrpE, Hsp18, Hsp90, HtrA, Map, TufA, TufB, or YacI, or functionally equivalent variants thereof, in any order. Other embodiments include nucleic acids encoding at least 3, 4, 5 or 6 of ClpB, ClpC, ClpP, DnaK, DnaJ, GreA, GroES, GroEL, GrpE, Hsp18, Hsp90, HtrA, Map, TufA, TufB, or YacI, or a functionally equivalent variant of any one or more thereof, in any order.

Exemplary amino acid sequences and nucleic acid sequence encoding each of the above enzymes is provided in GenBank as herein before described. However, skilled persons will readily appreciate alternative nucleic acids sequences encoding the enzymes or functionally equivalent variants thereof, having regard to the information contained herein, in GenBank and other databases, and the genetic code.

In one embodiment, the nucleic acid encodes both GroES and GroEL. In one particular embodiment, the nucleic acid comprises SEQ_ID No 3 and 4, or functionally equivalent variants thereof, in any order. In one embodiment, the nucleic acid comprises SEQ ID_NO. 12, or a functionally equivalent variant thereof.

In one embodiment, the nucleic acids of the invention will further comprise a promoter. Preferably, the promoter is as herein before described, and in a particular embodiment a pyruvate:ferredoxin oxidoreductase promoter. In one particular embodiment, the promoter has the nucleic acid sequence of SEQ_ID NO. 5 or a functionally equivalent variant thereof.

The nucleic acids of the invention may remain extra-chromosomal upon transformation of a parental microorganism or may be adapted for intergration into the genome of the microorganism. Accordingly, nucleic acids of the invention may include additional nucleotide sequences adapted to assist integration (for example, a region which allows for homologous recombination and targeted integration into the host genome) or stable expression and replication of an extrachromosomal construct (for example, origin of replication, promoter and other regulatory sequences).

In one embodiment, the nucleic acid is nucleic acid construct or vector. In one particular embodiment, the nucleic acid construct or vector is an expression construct or vector, however other constructs and vectors, such as those used for cloning are encompassed by the invention. In one particular embodiment, the expression construct or vector is a plasmid.

In one particular embodiment, the invention provides an expression construct or vector comprising a nucleic acid sequence encoding at least one enzyme, preferable two or more enzymes, which when expressed in a microorganism, results in the microorganism having an increased tolerance to ethanol. Preferably, the enzymes are as referred to herein before.

In one embodiment, the expression construct/vector comprises nucleic acid sequences encoding each of GroES (SEQ ID No. 1) and GroEL (SEQ_ID NO. 2). In one particular embodiment, the expression construct/vector comprises the nucleic acid sequences SEQ_ID NO. 3 and 4 or a functionally equivalent variant thereof, in any order. In one embodiment, the expression construct/vector comprises SEQ ID_NO. 12, or a functionally equivalent variant thereof.

Preferably the expression construct/vector will further comprise a promoter, as herein before described. In one embodiment, the promoter allows for constitutive expression of the genes under its control. However, inducible promoters may also be employed. It will be appreciated by those of skill in the art that other promoters which can direct expression, preferably a high level of expression under appropriate fermentation conditions, would be effective as alternatives to the presently preferred embodiments.

It will be appreciated that an expression construct/vector of the present invention may contain any number of regulatory elements in addition to the promoter as well as additional genes suitable for expression of further proteins if desired. In one embodiment the expression construct/vector includes one promoter. In another embodiment, the expression construct/vector includes two or more promoters. In one particular embodiment, the expression construct/vector includes one promoter for each gene to be expressed. In one embodiment, the expression construct/vector includes one or more ribosomal binding sites, preferably a ribosomal binding site for each gene to be expressed.

It will be appreciated by those of skill in the art that the nucleic acid sequences and construct/vector sequences described herein may contain standard linker nucleotides such as those required for ribosome binding sites and/or restriction sites. Such linker sequences should not be interpreted as being required and do not provide a limitation on the sequences defined.

In one particular embodiment of the invention, the expression construct/vector is an expression plasmid comprising the nucleotide sequence SEQ ID No. 17.

The invention also provides nucleic acids which are capable of hybridising to at least a portion of a nucleic acid herein described, a nucleic acid complementary to any one thereof, or a functionally equivalent variant of any one thereof. Such nucleic acids will preferably hybridise to such nucleic acids, a nucleic acid complementary to any one thereof, or a functionally equivalent variant of any one thereof, under stringent hybridisation conditions. “Stringent hybridisation conditions” means that the nucleic acid is capable of hybridising to a target template under standard hybridisation conditions such as those described in Sambrook et al, Molecular Cloning: A Laboratory Manual (1989), Cold Spring Harbor Laboratory Press, New York, USA. It will be appreciated that the minimal size of such nucleic acids is a size which is capable of forming a stable hybrid between a given nucleic acid and the complementary sequence to which it is designed to hybridise. Accordingly, the size is dependent on the nucleic acid composition and percent homology between the nucleic acid and its complementary sequence, as well as the hybridisation conditions which are utilised (for example, temperature and salt concentrations). In one embodiment, the nucleic acid is at least 10 nucleotides in length, at least 15 nucleotides in length, at least, 20 nucleotides in length, at least 25 nucleotides in length, or at least 30 nucleotides in length.

In one embodiment the invention provides a nucleic acid consisting of the sequence of any one of SEQ ID NO.s 6, 7, 8, 9, 10, and 11.

Nucleic acids and nucleic acid constructs, including the expression construct/vector of the invention may be constructed using any number of techniques standard in the art. For example, chemical synthesis or recombinant techniques may be used. Such techniques are described, for example, in Sambrook et al (Molecular Cloning: A laboratory manual, Cold Spring Harbor Laboratory Press, Cold Spring Harbor, NY, 1989). Further exemplary techniques are described in the Examples section herein after. Essentially, the individual genes and regulatory elements will be operably linked to one another such that the genes can be expressed to form the desired proteins. Suitable vectors for use in the invention will be appreciated by those of ordinary skill in the art. However, by way of example, the following vectors may be suitable: pMTL80000 shuttle vectors, pIMP1, pJIR750 and the plasmids exemplified in the Examples section herein after.

It should be appreciated that nucleic acids of the invention may be in any appropriate form, including RNA, DNA, or cDNA, including double-stranded and single-stranded nucleic acids.

The invention also provides host organisms, particularly microorganisms, and including viruses, bacteria, and yeast, comprising any one or more of the nucleic acids described herein.

The one or more exogenous nucleic acids may be delivered to a parental microorganism as naked nucleic acids or may be formulated with one or more agents to facilitate the tranformation process (for example, liposome-conjugated nucleic acid, an organism in which the nucleic acid is contained). The one or more nucleic acids may be DNA, RNA, or combinations thereof, as is appropriate.

The microorganisms of the invention may be prepared from a parental microorganism and one or more exogenous nucleic acids using any number of techniques known in the art for producing recombinant microorganisms. By way of example only, transformation (including transduction or transfection) may be achieved by electroporation, conjugation, or chemical and natural competence. Suitable transformation techniques are described for example in Sambrook J, Fritsch E F, Maniatis T: Molecular Cloning: A laboratory Manual, Cold Spring Harbour Labrotary Press, Cold Spring Harbour, 1989.

In certain embodiments, due to the restriction systems which are active in the microorganism to be transformed, it is necessary to methylate the nucleic acid to be introduced into the microorganism. This can be done using a variety of techniques, including those described below, and further exemplified in the Examples section herein after.

By way of example, in one embodiment, a recombinant microorganism of the invention is produced by a method comprises the following steps:

introduction into a shuttle microorganism of (i) of an expression construct/vector as described herein and (ii) a methylation construct/vector comprising a methyltransferase gene; expression of the methyltransferase gene;
isolation of one or more constructs/vectors from the shuttle microorganism; and, introduction of the one or more construct/vector into a destination microorganism.

In one embodiment, the methyltransferase gene of step B is expressed consitutively. In another embodiment, expression of the methyltransferase gene of step B is induced.

The shuttle microorganism is a microorganism, preferably a restriction negative microorganism, that facilitates the methylation of the nucleic acid sequences that make up the expression construct/vector. In a particular embodiment, the shuttle microorganism is a restriction negative E. coli or Bacillus subtillis.

The methylation construct/vector comprises a nucleic acid sequence encoding a methyltransferase.

Once the expression construct/vector and the methylation construct/vector are introduced into the shuttle microorganism, the methyltransferase gene present on the methylation construct/vector in induced. Induction may be by any suitable promoter system although in one particular embodiment of the invention, the methylation construct/vector comprises an inducible lac promoter (preferably encoded by SEQ_ID NO 19) and is induced by addition of lactose or an analogue thereof, more preferably isopropyl-β-D-thio-galactoside (IPTG). Other suitable promoters include the ara, tet, or T7 system. In a further embodiment of the invention, the methylation construct/vector promoter is a constitutive promoter.

In a particular embodiment, the methylation construct/vector has an origin of replication specific to the identity of the shuttle microorganism so that any genes present on the methylation construct/vector are expressed in the shuttle microorganism. Preferably, the expression construct/vector has an origin of replication specific to the identity of the destination microorganism so that any genes present on the expression construct/vector are expressed in the destination microorganism.

Expression of the methyltransferase enzyme results in methylation of the genes present on the expression construct/vector. The expression construct/vector may then be isolated from the shuttle microorganism according to any one of a number of known methods. By way of example only, the methodology described in the Examples section described hereinafter may be used to isolate the expression construct/vector.

In one particular embodiment, both construct/vector are concurrently isolated.

The expression construct/vector may be introduced into the destination microorganism using any number of known methods. However, by way of example, the methodology described in the Examples section hereinafter may be used. Since the expression construct/vector is methylated, the nucleic acid sequences present on the expression construct/vector are able to be incorporated into the destination microorganism and successfully expressed.

It is envisaged that a methyltransferase gene may be introduced into a shuttle microorganism and over-expressed. Thus, in one embodiment, the resulting methyltransferase enzyme may be collected using known methods and used in vitro to methylate an expression plasmid. The expression construct/vector may then be introduced into the destination microorganism for expression. In another embodiment, the methyltransferase gene is introduced into the genome of the shuttle microorganism followed by introduction of the expression construct/vector into the shuttle microorganism, isolation of one or more constructs/vectors from the shuttle microorganism and then introduction of the expression construct/vector into the destination microorganism.

It is envisaged that the expression construct/vector and the methylation construct/vector as defined above may be combined to provide a composition of matter. Such a composition has particular utility in circumventing restriction barrier mechanisms to produce the recombinant microorganisms of the invention.

In one particular embodiment, the expression construct/vector and/or the methylation construct/vector are plasmids.

Skilled person will appreciate a number of suitable methyltransferases of use in producing the microorganisms of the invention. However, by way of example the Bacillus subtilis phage φT1 methyltransferase and the methyltransferase described in the Examples herein after may be used. Nucleic acids encoding suitable methyltransferases will be readily appreciated having regard to the sequence of the desired methyltransferase and the genetic code. In one embodiment, the nucleic acid encoding a methyltransferase is described in the Examples herein after (for example the nucleic acid of SEQ_ID NO. 28.

Any number of constructs/vectors adapted to allow expression of a methyltransferase gene may be used to generate the methylation construct/vector. However, by way of example, the plasmid described in the Examples section hereinafter may be used. In one particular embodiment, the plasmid has the sequence of SEQ_ID NO. 19.

The invention provides a method for the production ethanol or one or more other products by microbial fermentation comprising fermenting a substrate comprising CO using a recombinant microorganism of the invention. The methods of the invention may be used to reduce the total atmospheric carbon emissions from an industrial process.

Preferably, the fermentation comprises the steps of anaerobically fermenting a substrate in a bioreactor to produce ethanol, or ethanol and one or more other products using a recombinant microorganism of the invention.

In one embodiment the method comprises the steps of:
(a) providing a substrate comprising CO to a bioreactor containing a culture of one or more microorganism of the first aspect of the invention; and
(b) anaerobically fermenting the culture in the bioreactor to produce one or more products including ethanol.

In one embodiment the method comprises the steps of:
(a) capturing CO-containing gas produced as a result of the industrial process, before the gas is released into the atmosphere;
(b) the anaerobic fermentation of the CO-containing gas to produce one or more products including ethanol by a culture containing one or more microorganism of the first aspect of the invention.

In one embodiment, the ethanol concentration in the fermentation broth is at least approximately 5.5% by weight. In another embodiment, the ethanol concentration in the fermentation broth is at least approximately 6% by weight.

In an embodiment of the invention, the gaseous substrate fermented by the microorganism is a gaseous substrate containing CO. The gaseous substrate may be a CO-containing waste gas obtained as a by-product of an industrial process, or from some other source such as from automobile exhaust fumes. In certain embodiments, the industrial process is selected from the group consisting of ferrous metal products manufacturing, such as a steel mill, non-ferrous products manufacturing, petroleum refining processes, gasification of coal, electric power production, carbon black production, ammonia production, methanol production and coke manufacturing. In these embodiments, the CO-containing gas may be captured from the industrial process before it is emitted into the atmosphere, using any convenient method. The CO may be a component of syngas (gas comprising carbon monoxide and hydrogen). The CO produced from industrial processes is normally flared off to produce CO₂ and therefore the invention has particular utility in reducing CO₂ greenhouse gas emissions and producing butanol for use as a biofuel. Depending on the composition of the gaseous CO-containing substrate, it may also be desirable to treat it to remove any undesired impurities, such as dust particles before introducing it to the fermentation. For example, the gaseous substrate may be filtered or scrubbed using known methods.

It will be appreciated that for growth of the bacteria and CO-to-ethanol (and/or other product(s)) to occur, in addition to the CO-containing substrate gas, a suitable liquid nutrient medium will need to be fed to the bioreactor. The substrate and media may be fed to the bioreactor in a continuous, batch or batch fed fashion. A nutrient medium will contain vitamins and minerals sufficient to permit growth of the micro-organism used. Anaerobic media suitable for fermentation to produce ethanol (and optionally one or more other products) using CO are known in the art. For example, suitable media are described in Biebel (Journal of Industrial Microbiology & Biotechnology (2001) 27, 18-26). The substrate and media may be fed to the bioreactor in a continuous, batch or batch fed fashion. In one embodiment of the invention the media is as described in the Examples section herein after.

The fermentation should desirably be carried out under appropriate conditions for the CO-to-ethanol (and/or other product(s)) fermentation to occur. Reaction conditions that should be considered include pressure, temperature, gas flow rate, liquid flow rate, media pH, media redox potential, agitation rate (if using a continuous stirred tank reactor), inoculum level, maximum gas substrate concentrations to ensure that CO in the liquid phase does not become limiting, and maximum product concentrations to avoid product inhibition.

In addition, it is often desirable to increase the CO concentration of a substrate stream (or CO partial pressure in a gaseous substrate) and thus increase the efficiency of fermentation reactions where CO is a substrate. Operating at increased pressures allows a significant increase in the rate of CO transfer from the gas phase to the liquid phase where it can be taken up by the micro-organism as a carbon source for the production of ethanol (and/or other product(s)). This in turn means that the retention time (defined as the liquid volume in the bioreactor divided by the input gas flow rate) can be reduced when bioreactors are maintained at elevated pressure rather than atmospheric pressure. The optimum reaction conditions will depend partly on the particular micro-organism of the invention used. However, in general, it is preferred that the fermentation be performed at pressure higher than ambient pressure. Also, since a given CO-to-ethanol (and/or other product(s)) conversion rate is in part a function of the substrate retention time, and achieving a desired retention time in turn dictates the required volume of a bioreactor, the use of pressurized systems can greatly reduce the volume of the bioreactor required, and consequently the capital cost of the fermentation equipment. According to examples given in U.S. Pat. No. 5,593,886, reactor volume can be reduced in linear proportion to increases in reactor operating pressure, i.e. bioreactors operated at 10 atmospheres of pressure need only be one tenth the volume of those operated at 1 atmosphere of pressure.

The benefits of conducting a gas-to-ethanol fermentation at elevated pressures has been described elsewhere. For example, WO 02/08438 describes gas-to-ethanol fermentations performed under pressures of 30 psig and 75 psig, giving ethanol productivities of 150 g/l/day and 369 g/l/day respectively. However, example fermentations performed using similar media and input gas compositions at atmospheric pressure were found to produce between 10 and 20 times less ethanol per litre per day.

It is also desirable that the rate of introduction of the CO-containing gaseous substrate is such as to ensure that the concentration of CO in the liquid phase does not become limiting. This is because a consequence of CO-limited conditions may be that the ethanol product is consumed by the culture.

The composition of gas streams used to feed a fermentation reaction can have a significant impact on the efficiency and/or costs of that reaction. For example, O2 may reduce the efficiency of an anaerobic fermentation process. Processing of unwanted or unnecessary gases in stages of a fermentation process before or after fermentation can increase the burden on such stages (e.g. where the gas stream is compressed before entering a bioreactor, unnecessary energy may be used to compress gases that are not needed in the fermentation). Accordingly, it may be desirable to treat substrate streams, particularly substrate streams derived from industrial sources, to remove unwanted components and increase the concentration of desirable components.

In certain embodiments a culture of a bacterium of the invention is maintained in an aqueous culture medium. Preferably the aqueous culture medium is a minimal anaerobic microbial growth medium. Suitable media are known in the art and described for example in U.S. Pat. Nos. 5,173,429 and 5,593,886 and WO 02/08438, and as described in the Examples section herein after.

Ethanol, or a mixed alcohol stream containing ethanol and one or more other alcohols, or a mixed product stream comprising ethanol and/or one or more other products, may be recovered from the fermentation broth by methods known in the art, such as fractional distillation or evaporation, pervaporation, and extractive fermentation, including for example, liquid-liquid extraction. By-products such as acids including acetate may also be recovered from the fermentation broth using methods known in the art. For example, an adsorption system involving an activated charcoal filter or electrodialysis may be used. Alternatively, continuous gas stripping may also be used.

In certain preferred embodiments of the invention, ethanol and/or one or more other products are recovered from the fermentation broth by continuously removing a portion of the broth from the bioreactor, separating microbial cells from the broth (conveniently by filtration), and recovering one or more products from the broth. Alcohols may conveniently be recovered for example by distillation, and acids may be recovered for example by adsorption on activated charcoal. The separated microbial cells are preferably returned to the fermentation bioreactor. The cell free permeate remaining after any alcohol(s) and acid(s) have been removed is also preferably returned to the fermentation bioreactor. Additional nutrients (such as B vitamins) may be added to the cell free permeate to replenish the nutrient medium before it is returned to the bioreactor.

Also, if the pH of the broth was adjusted as described above to enhance adsorption of acetic acid to the activated charcoal, the pH should be re-adjusted to a similar pH to that of the broth in the fermentation bioreactor, before being returned to the bioreactor.

### Examples

The invention will now be described in more detail with reference to the following non-limiting examples.

### Microorganism

The following work was conducted using Clostridium autoethanogenum DSM23693 (DSMZ (The German Collection of Microorganisms and Cell Cultures), Inhoffenstrage 7 B, 38124 Braunschweig, GERMANY.

### Ethanol Tolerance of Clostridium Autoethanogenum

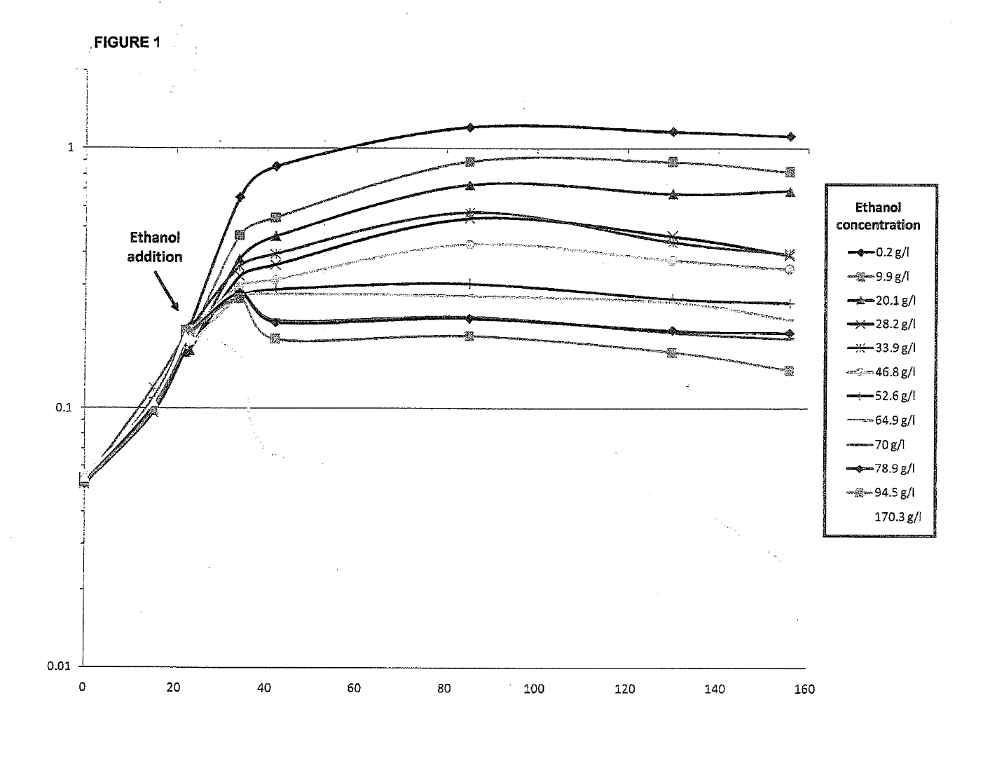

The ethanol tolerance of Clostridium autoethanogenum DSM23693 was tested in serum bottles (FIG. 1). Growth was found to be inhibited at concentrations between 10-20 g/l ethanol, while growth completely ceased after addition of >50 g/l or >5% ethanol.

Ethanol was added in various concentrations to an active growing culture at 37° C. in PETC medium (Table 1) with 30 psi steel mill gas as substrate. The media was prepared by using standard anaerobic techniques (Hungate R E. A roll tube method for cultivation of strict anaerobes, In Norris J R and Ribbons D W (eds.), Methods in Microbiology, vol. 3B. Academic Press, NY, 1969: 117-132; Breznak JA and Costilow RN, Physicochemical factors in growth, In Gerhardt P (ed.), Methods for general and molecular bacteriology. American Society for Microbiology, Washington, 1994: 137-154). Ethanol concentrations were confirmed by HPLC analysis using an Agilent 1100 Series HPLC system equipped with a RID (Refractive Index Detector) operated at 35° C. and an Alltech IOA-2000 Organic acid column (150×6.5 mm, particle size 5 μm) kept at 60° C. Slightly acidified water was used (0.005 M H₂50₄) as mobile phase with a flow rate of 0.7 ml/min. To remove proteins and other cell residues, 400 μl samples were mixed with 100 μl of a 2% (w/v) 5-Sulfosalicylic acid and centrifuged at 14,000×g for 3 min to separate precipitated residues. 10 μl of the supernatant were then injected into the HPLC for analyses.

TABLE 1
PETC medium

| Media component | Concentration per 1.0 L of media | |
|---|---|---|
| NH₄Cl | 1 | g |
| KCl | 0.1 | g |
| MgSO₄•7H₂O | 0.2 | g |
| NaCl | 0.8 | g |
| KH₂PO₄ | 0.1 | g |
| CaCl₂ | 0.02 | g |
| Trace metal solution (see below) | 10 | ml |
| Wolfe's vitamin solution (see below) | 10 | ml |
| Yeast Extract | 1 | g |
| Resazurin (2 g/L stock) | 0.5 | ml |
| NaHCO₃ | 2 | g |
| Reducing agent | 0.006-0.008% (v/v) | |
| Wolfe's vitamin solution | per L of Stock | |
| Biotin | 2 | mg |
| Folic acid | 2 | mg |
| Pyridoxine hydrochloride | 10 | mg |
| Thiamine•HCl | 5 | mg |
| Riboflavin | 5 | mg |
| Nicotinic acid | 5 | mg |
| Calcium D-(+)-pantothenate | 5 | mg |
| Vitamin B₁₂ | 0.1 | mg |
| p-Aminobenzoic acid | 5 | mg |
| Thioctic acid | 5 | mg |
| Trace metal solution | per L of stock | |
| Nitrilotriacetic Acid | 2 | g |
| MnSO₄•H₂O | 1 | g |
| Fe (SO₄)₂(NH₄)₂•6H₂O | 0.8 | g |
| CoCl₂•6H₂O | 0.2 | g |
| ZnSO₄•7H₂O | 0.2 | mg |
| CuCl₂•2H₂O | 0.02 | g |
| NaMoO₄•2H₂O | 0.02 | g |
| Na₂SeO₃ | 0.02 | g |
| NiCl₂•6H₂O | 0.02 | g |
| Na₂WO₄•2H₂O | 0.02 | g |
| Reducing agent stock | per 100 mL of stock | |
| NaOH | 0.9 | g |
| Cystein•HCl | 4 | g |
| Na₂S | 4 | g |

Genetic Modification of Clostridium Autoethanogenum for improved Ethanol Tolerance

Ethanol concentrations greater than 50 g/l or 5% have been shown to inhibit the growth of Clostridium autoethanogenum completely (FIG. 1) and thus form a physical limit for the production of ethanol. Heat shock protein/chaperonin GroES (SEQ_ID NO. 1) and GroEL (SEQ_ID NO. 2) were overproduced in Clostridium autoethanogenum DSM23693, which conferred higher tolerance of Clostridium autoethanogenum to ethanol.

### Promoter for Gene Overexpression:

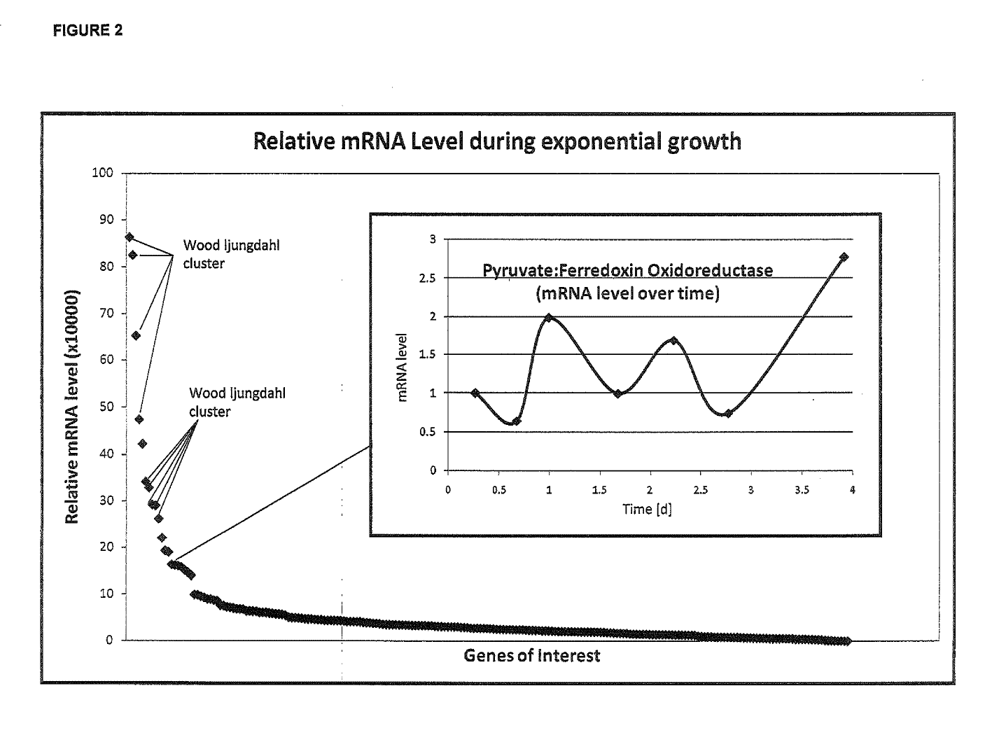

For overexpression of genes groES (SEQ_ID NO. 3) and groEL (SEQ_ID NO. 4), a strong native pyruvate:ferredoxin oxidoreductase promoter was used. This gene was found to be constitutively expressed at a high level (FIG. 2).

### Amplification of Genes and Promoter Sequences:

Standard Recombinant DNA and molecular cloning techniques were used in this invention (Sambrook J, Fritsch E F, Maniatis T: Molecular Cloning: A laboratory Manual, Cold Spring Harbour Labrotary Press, Cold Spring Harbour, 1989; Ausubel F M, Brent R, Kingston R E, Moore D D, Seidman J G, Smith J A, Struhl K: Current protocols in molecular biology. John Wiley & Sons, Ltd., Hoboken, 1987). DNA sequences of groES and groEL genes and pyruvate:ferredoxin oxidoreductase (P_{pfor}) were sequenced from C. autoethanogenum (Table 2).

TABLE 2
Gene sequences

| Gene/Promoter | Description | SEQ ID NO |
|---|---|---|
| groES | Clostridium autoethanogenum | 3 |
| groEL | Clostridium autoethanogenum | 4 |
| Pyruvate: ferredoxin | Clostridium autoethanogenum | 5 |
| oxidoreductase | | |
| promoter (P_{PFOR}) | | |

Genomic DNA from Clostridium autoethanogenum DSM23693 was isolated using a modified method by Bertram and Dürre (1989), 1989 (Conjugal transfer and expression of streptococcal transposons in Clostridium acetobutylicum. Arch Microbiol 151: 551-557). A 100-ml overnight culture was harvested (6,000×g, 15 min, 4° C.), washed with potassium phosphate buffer (10 mM, pH 7.5) and suspended in 1.9 ml STE buffer (50 mM Tris-HCl, 1 mM EDTA, 200 mM sucrose; pH 8.0). 300 μl lysozyme (˜100,000 U) was added and the mixture was incubated at 37° C. for 30 min, followed by addition of 280 μl of a 10% (w/v) SDS solution and another incubation for 10 min. RNA was digested at room temperature by addition of 240 μl of an EDTA solution (0.5 M, pH 8), 20 μl Tris-HCl (1 M, pH 7.5), and 10 μl RNase A (Fermentas Life Sciences). Then, 100 μl Proteinase K (0.5 U) was added and proteolysis took place for 1-3 h at 37° C. Finally, 600 μl of sodium perchlorate (5 M) was added, followed by a phenol-chloroform extraction and an isopropanol precipitation. DNA quantity and quality was inspected spectrophotometrically.

All sequences were amplified from isolated genomic DNA by PCR with oligonucleotides given in Table 3 using iProof High Fidelity DNA Polymerase (Bio-Rad Labratories) and the following program: initial denaturation at 98° C. for 30 seconds, followed by 32 cycles of denaturation (98° C. for 10 seconds), annealing (50-62° C. for 30-120 seconds) and elongation (72° C. for 30-90 seconds), before a final extension step (72° C. for 10 minutes).

TABLE 3
Oligonucleotides for cloning

| | Oligonucleotide | | SEQ_ID |
|---|---|---|---|
| Target | Name | DNA Sequence (5' to 3') | NO. |
| groESL operon | SOE-GroESL-a- | GGGTTCATATGAAAATTAGACCACTTGG | 6 |
| | Ndel | | |
| groESL operon | SOE-GroESL-b | TCCCATGTTTTCATAAGGATCTTCTAATTC | 7 |
| groESL operon | SOE-GroESL-c | ATTAGAAGATCCTTATGAAAACATGGGAGC | 8 |
| groESL operon | SOE-GroESL-d- | CTTAGAATTCCTTTTGAATTAGTACATTCC | 9 |
| | EcoRI | | |
| Pyruvate: ferredoxin | Ppfor-NotI-F | AAGCGGCCGCAAAATAGTTGATAATAATGC | 10 |
| oxidoreductase | | | |
| promoter (P_{pfor}) | | | |
| Pyruvate: ferredoxin | Ppfor-Ndel-R | TACGCATATGAATTCCTCTCCTTTTCAAGC | 11 |
| oxidoreductase | | | |
| promoter (P_{pfor}) | | | |

Genes groES and groEL were found to form a common operon on the genome of Clostridium autoethanogenum. The whole operon was amplified by SOE (splicing by overlap extension) PCR (Heckman K L, Pease L R: Gene Splicing and Mutagenesis by PCR-Driven Overlap Extension. Nature Protocols 2007, 2: 924-932; Vallejo A N, Pogulis R J, Pease L R: In Vitro Synthesis of Novel Genes: Mutagenesis and Recombination by PCR. Genome Research 1994, 4: S123-S130) in order to mutate an obstructing NdeI restriction site (CTTATG for CTGATG) within the groEL gene while retaining the same amino acid sequence (SEQ_ID NO. 12).

Initial PCRs using internal primer pairs “SOE-GroESL-a-NdeI” (SEQ_ID NO. 6) plus “SOE-GroESL-b” (SEQ_ID NO. 7) and “SOE-GroESL-c” (SEQ_ID NO. 8) plus “SOE-GroESL-d-EcoRI” (SEQ_ID NO. 9) generated overlapping fragments with complementary 3′ ends and a mutated NdeI site. These intermediate segments were then used as template for a second PCR using flanking oligonucleotides “SOE-GroESL-a-NdeI” (SEQ_ID NO. 6) and “SOE-GroESL-d-EcoRI” (SEQ_ID NO. 9) to create the full length product of the groESL operon without internal NdeI site (SEQ_ID NO. 12).

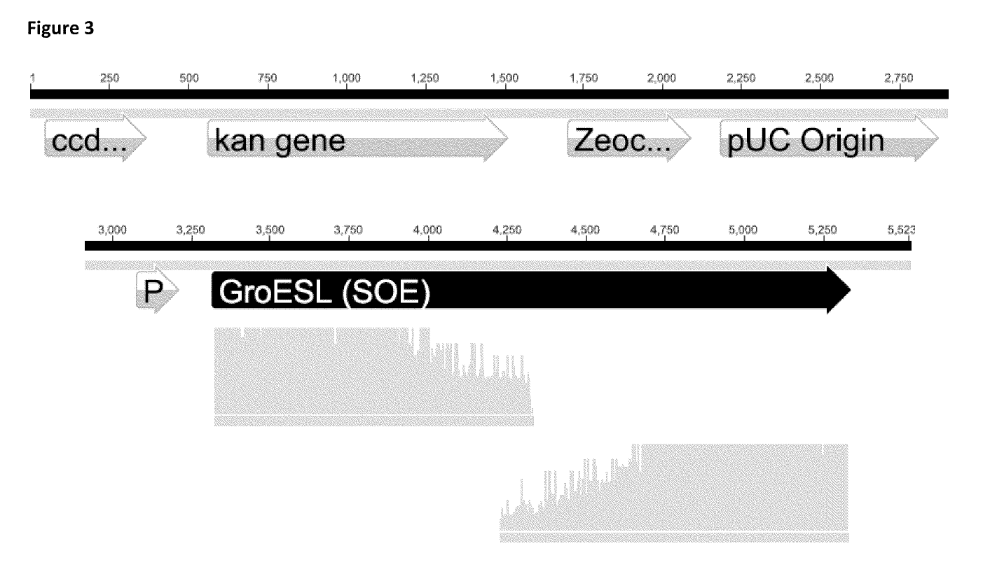

The PCR product was then cloned into vector pCR-Blunt II-TOPO, forming plasmid pCR-Blunt-GroESL, using Zero Blunt TOPO PCR cloning kit (Invitrogen) and E. coli strain DH5a-T1^{R} (Invitrogen). DNA sequencing using oligonucleotides M13 Forward (-20) (SEQ_ID NO. 13) and M13 Reverse (SEQ_ID NO. 14) showed that the groESL insert was free of mutation and the internal NdeI site was successfully mutated (FIG. 3).

Construction of a groESL Overexpression Plasmid:

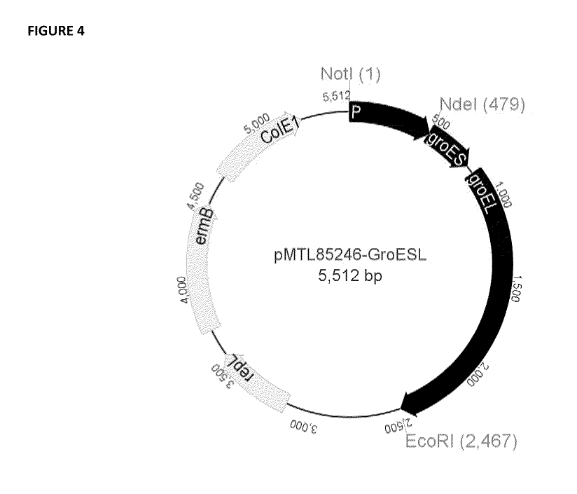

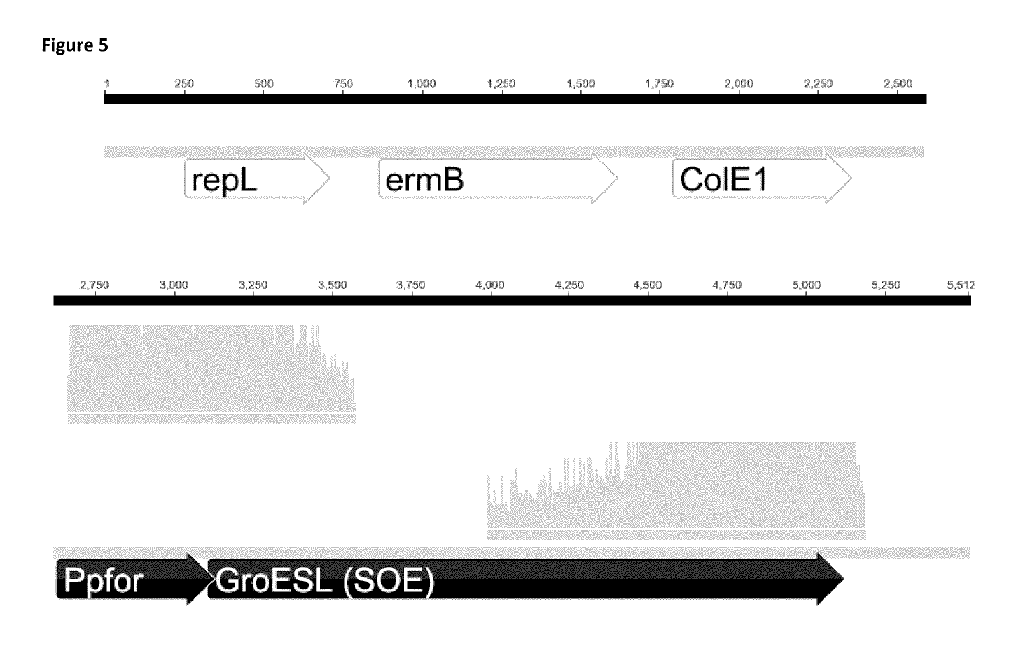

Construction of an expression plasmid was performed in E. coli DH5α-T1^{R} (Invitrogen). In a first step, the amplified pyruvate:ferredoxin oxidoreductase promoter region was cloned into the E. coli-Clostridium shuttle vector pMTL85141 (SEQ_ID NO. 15; FJ797651.1; Nigel Minton, University of Nottingham; Heap et al., 2009) using NotI and NdeI restriction sites, generating plasmid pMTL85146. As a second step, the antibiotic resistance marker was exchanged from catP to ermB (released from vector pMTL82254 (SEQ_ID NO. 16; FJ797646.1; Nigel Minton, University of Nottingham; Heap et al., 2009)) using restriction enzymes PmeI and FseI. The resulting plasmid pMTL85246 was then digested with NdeI and EcoRI and ligated with the groESL insert, which was released from plasmid pCR-Blunt-GroESL with NdeI and EcoRI, generating plasmid pMTL85246-GroESL (FIG. 4; SEQ_ID NO. 17). DNA sequencing using oligonucleotides M13 Forward (-20) (SEQ_ID NO. 13) and M13 Reverse (SEQ_ID NO. 14) confirmed successful cloning (FIG. 5).

### Methylation of DNA:

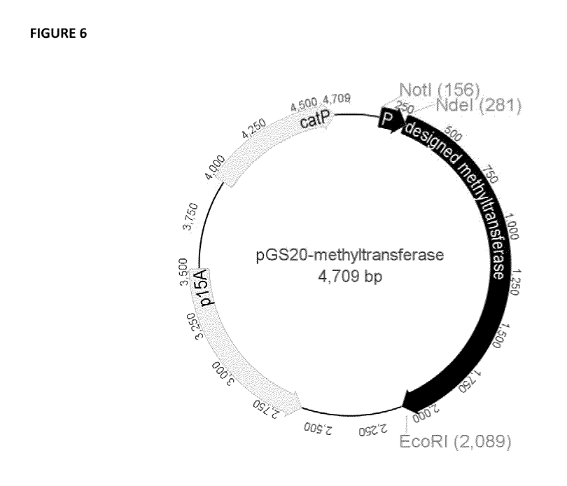

Transformation in Clostridium autoethanogenum DSM23693 is only possible with methylated DNA, due to the presence of various restriction systems. Methylation of plasmid DNA was created in vivo in the restriction negative E. coli strain XL1-blue MRF' with a plasmid encoded Type II methyltransferase (SEQ_ID NO. 18). The methyltransferase was design according the sequences of a methyltransferase of C. autoethanogenum, C. ragsdalei and C. ljungdahlii and then chemically synthesized and cloned into plasmid pGS20 (ATG:biosynthetics GmbH, Merzhausen, Germany) under control of an inducible lac promoter (FIG. 6; SEQ_ID NO. 19). Expression and methylation plasmid were co-transformed in E. coli and methylation induced by addition of 1 mM IPTG. Isolated plasmid mix (QIAGEN Plasmid Midi Kit; QIAGEN), was used for transformation, but only the expression plasmid pMTL85246-GroESL has a Gram-(+) replication origin.

Transformation of Expression Plasmid in C. autoethanogenum DSM23693 and C. Ljungdahlii DSM 13528:

Competent cells of C. autoethanogenum DSM23693 were made from a 50 ml culture grown in MES media (Table 4) and in presence of 40 mM threonine. At an OD₆₀₀ₙₘ of 0.4 (early to mid exponential growth phase), the cells were transferred into an anaerobic chamber and harvested at 4,700×g and 4° C. The culture was twice washed with ice-cold electroporation buffer (270 mM sucrose, 1 mM MgCl₂, 7 mM sodium phosphate, pH 7.4) and finally suspended in a volume of 500 μl fresh electroporation buffer. This mixture was transferred into a pre-cooled electroporation cuvette with a 0.4 cm electrode gap containing ˜1 μg of the methylated plasmid mix and 1 μl Type I restriction inhibitor (EPICENTRE). After a pulse (2.5 kV, 600 Ω, and 25 μF; time constant 4.5-4.7 ms) was applied using a Gene pulser Xcell electroporation system (Bio-Rad) the cells were regenerated for 8 hours in MES media and then plated on PETC media (Table 1) plates (1.2% Bacto™ Agar (Becton Dickinson) containing 4 μg /ml clarithromycin and 30 psi steel mill gas in the headspace. After 4-5 days, around 100 colonies were visible, which were used to inoculate selective liquid PETC media.

TABLE 4
MES media

| Media component | Concentration per 1.0 L of media | |
|---|---|---|
| NH₄Cl | 1 | g |
| KCl | 0.1 | g |
| MgSO₄•7H₂O | 0.2 | g |
| KH₂PO₄ | 0.2 | g |
| CaCl₂ | 0.02 | g |
| Trace metal solution (see Tab. 2) | 10 | ml |
| Wolfe's vitamin solution (see Tab. 2) | 10 | ml |
| Yeast Extract | 2 | g |
| Resazurin (2 g/L stock) | 0.5 | ml |
| 2-(N-morpholino)ethanesulfonic | 20 | g |
| acid (MES) | | |
| Reducing agent | 0.006-0.008% (v/v) | |
| Fructose | 5 | g |
| Sodium acetate | 0.25 | g |
| Fe (SO₄)₂(NH₄)₂•6H₂O | 0.05 | g |
| Nitriolotriacetic Acid | 0.05 | g |

| | |
|---|---|
| pH 5.7 | Adjusted with NaOH |

### Confirmation of Transformation Success:

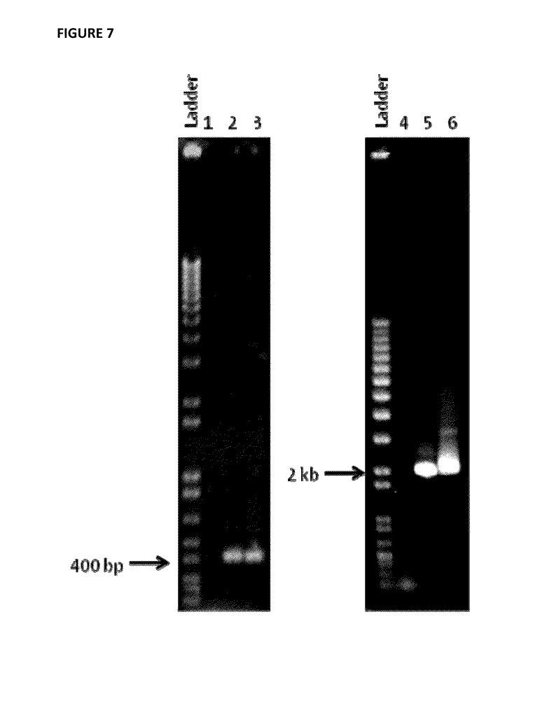

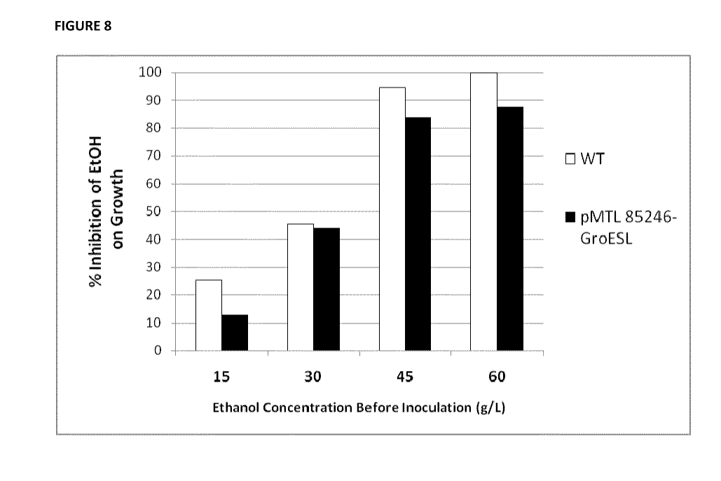

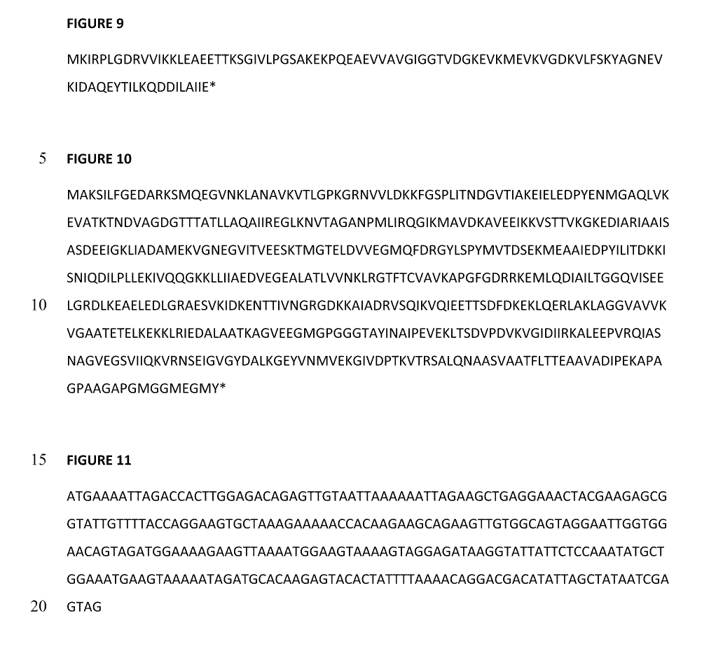

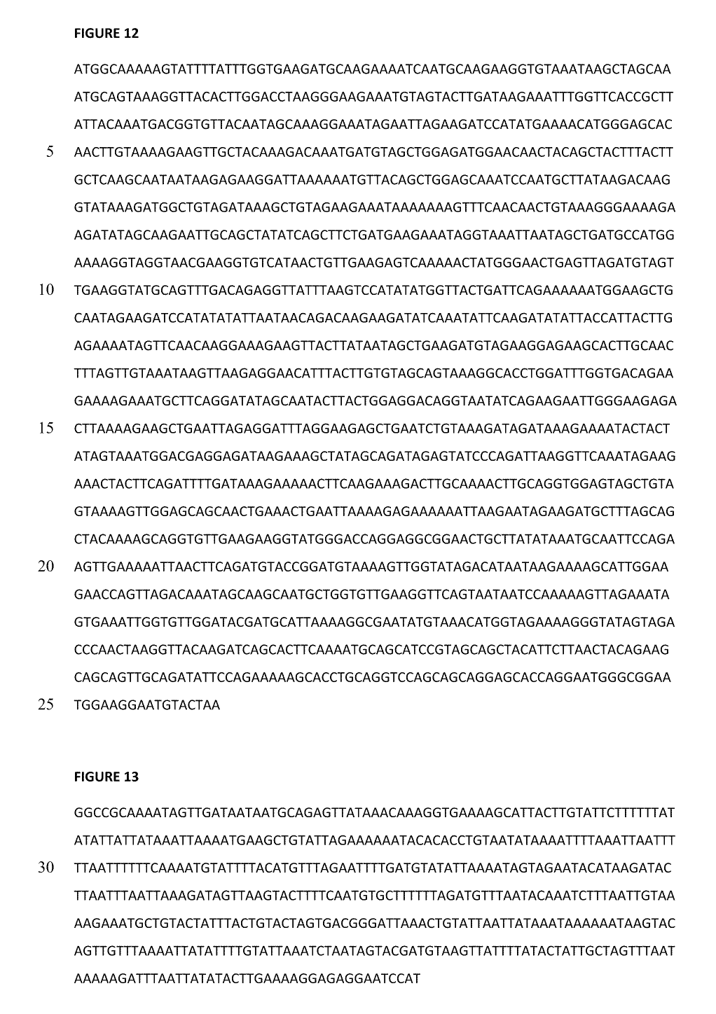

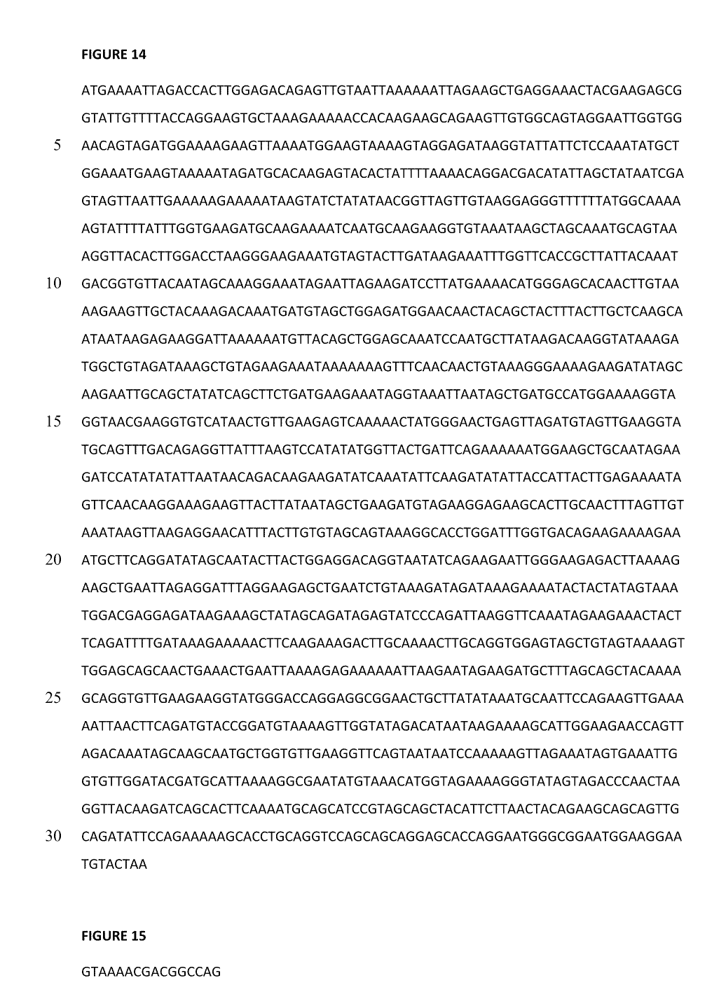

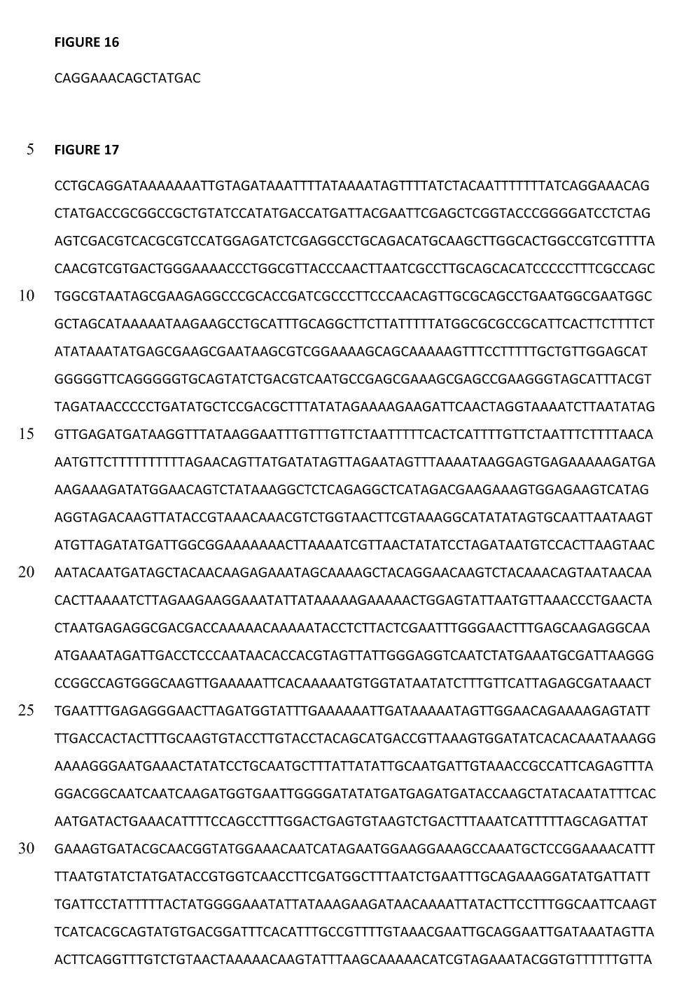

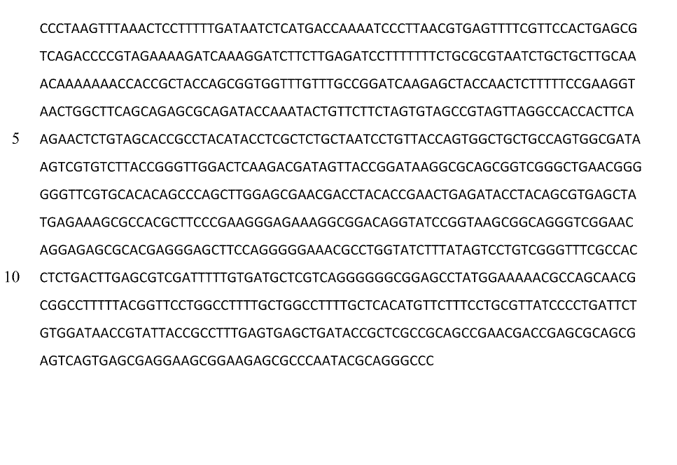

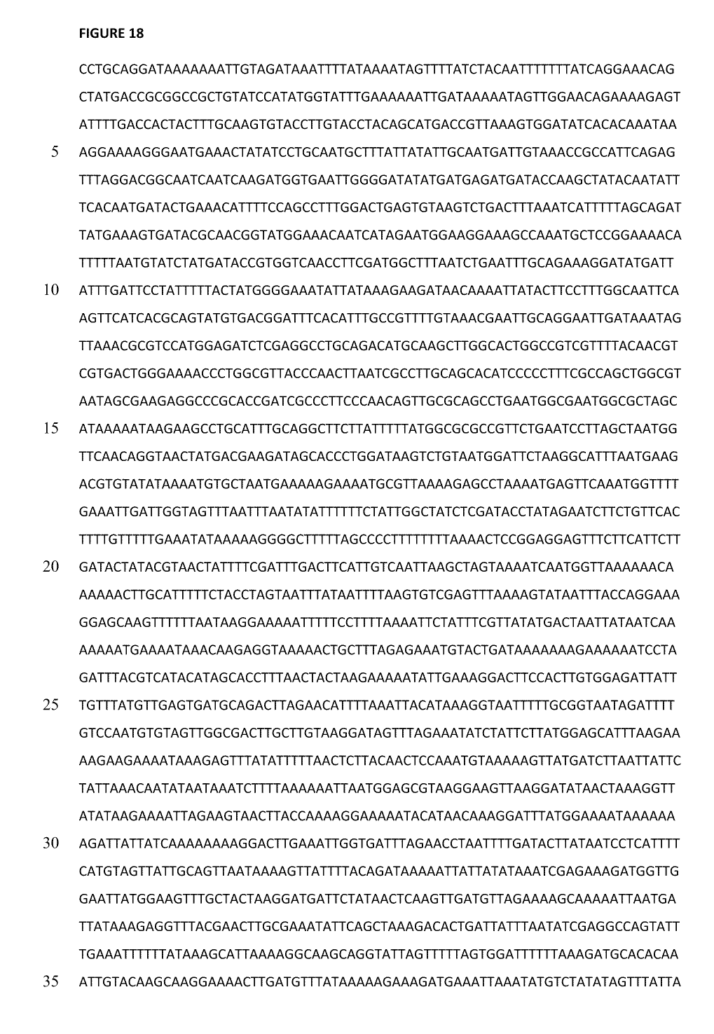

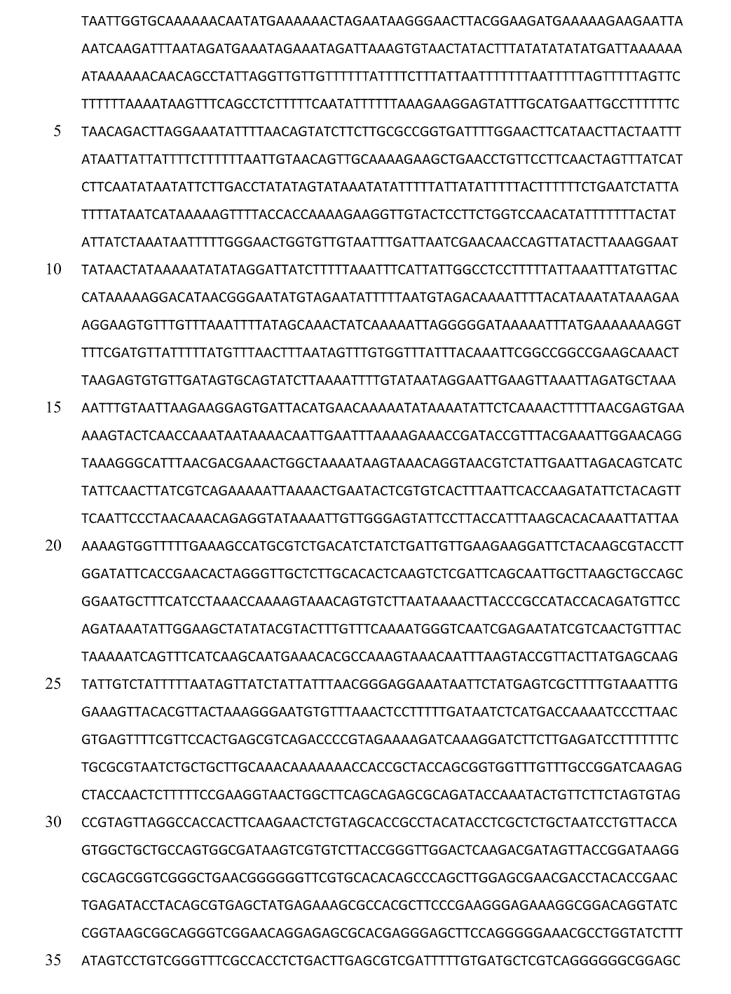

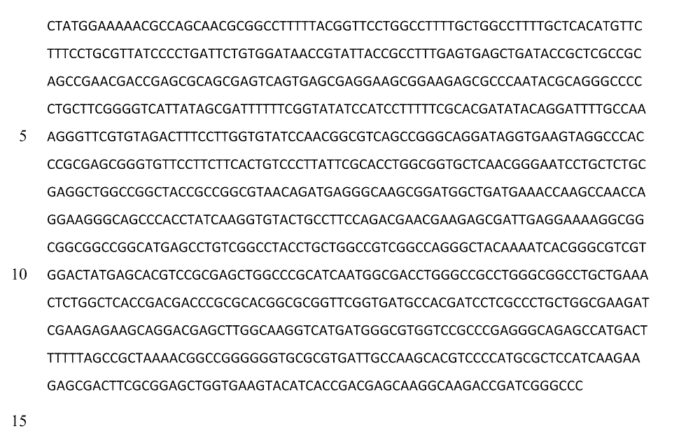

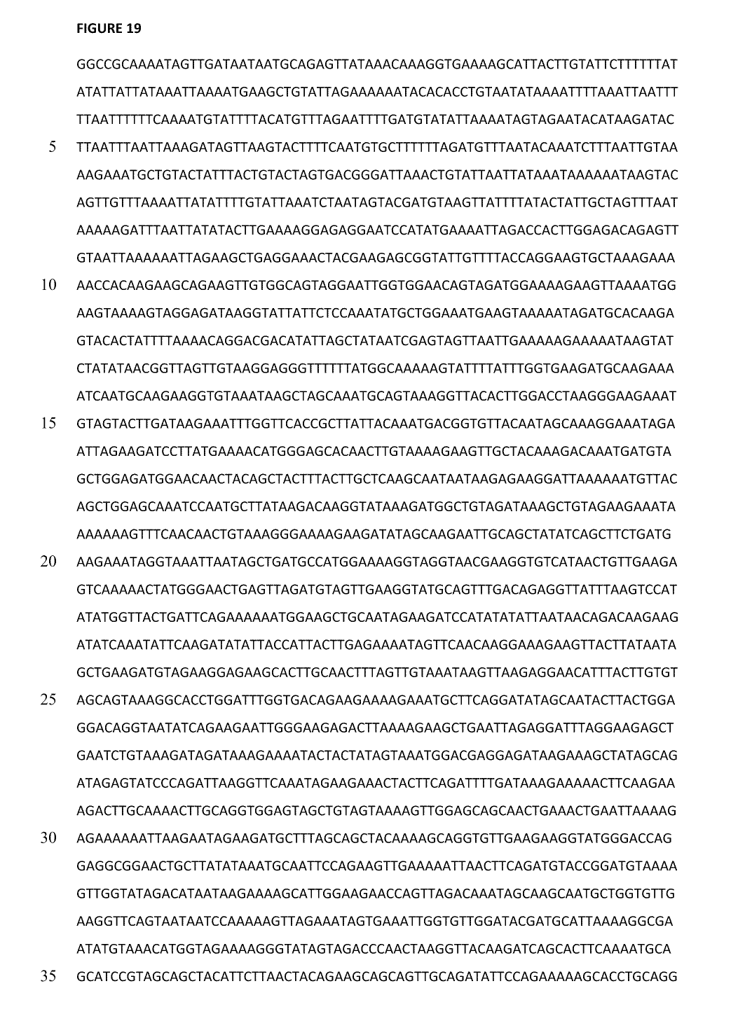

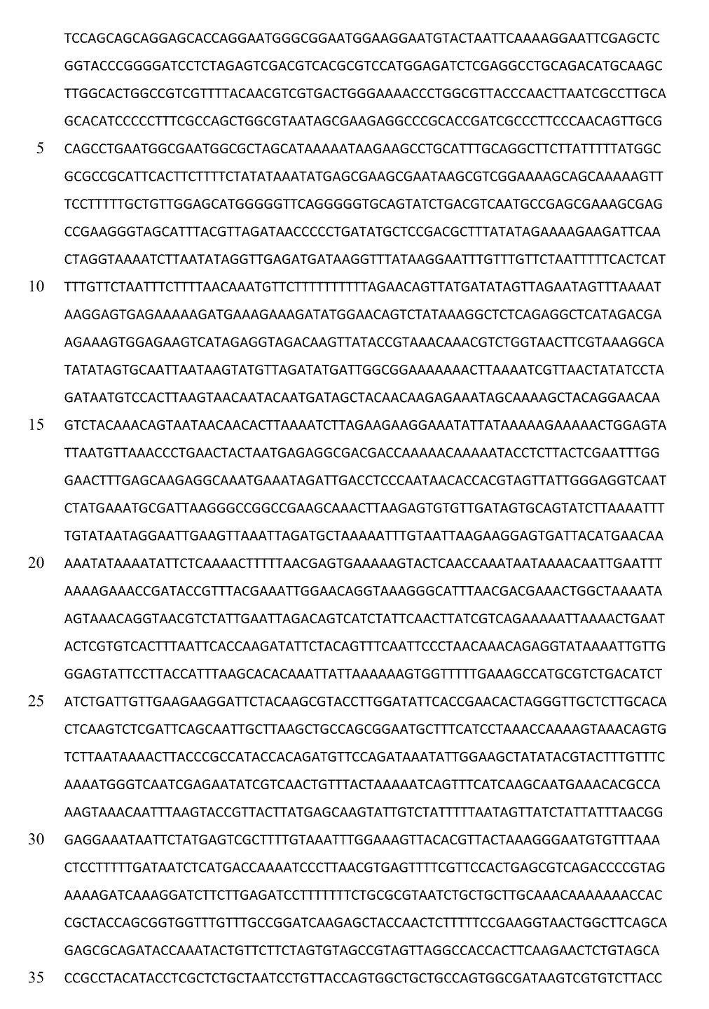

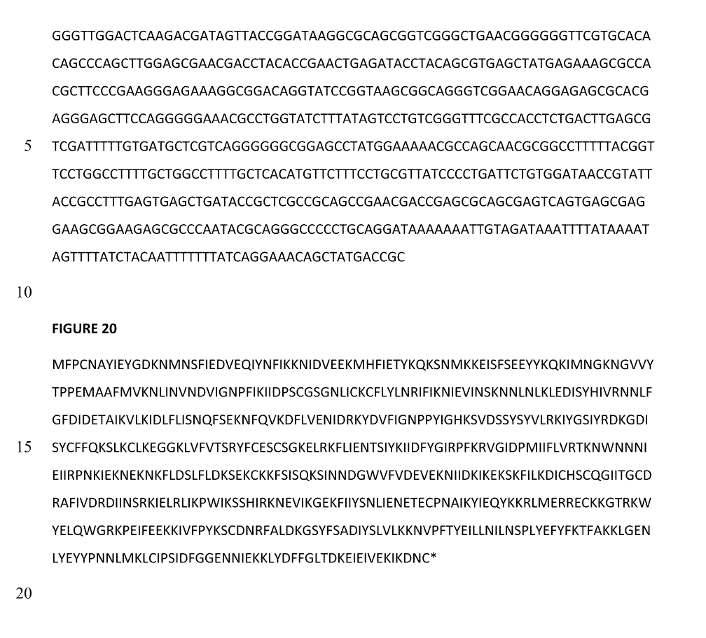

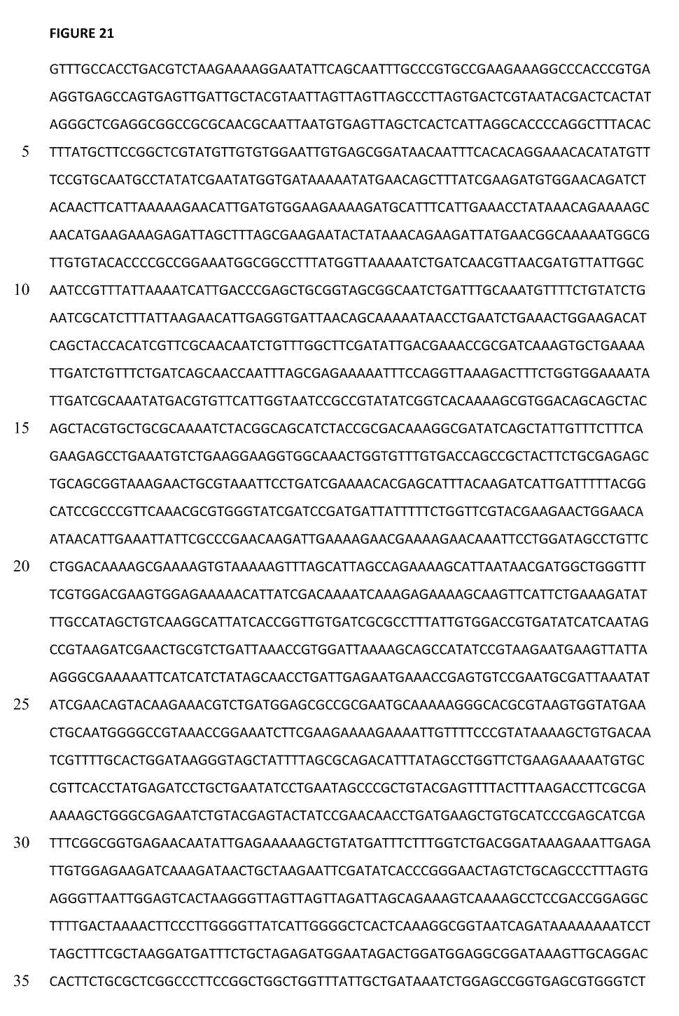

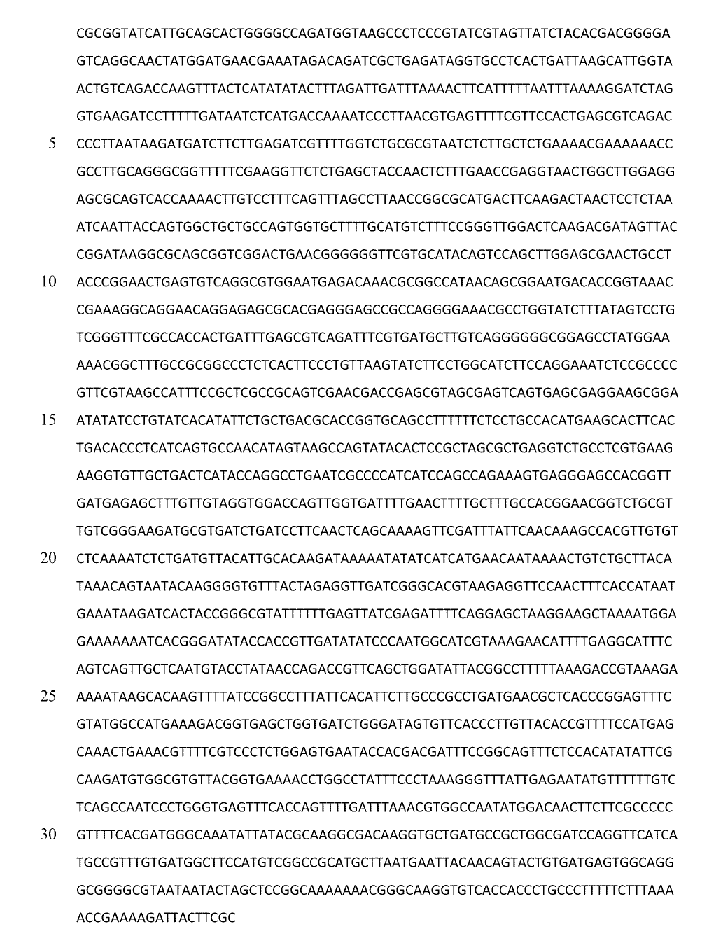

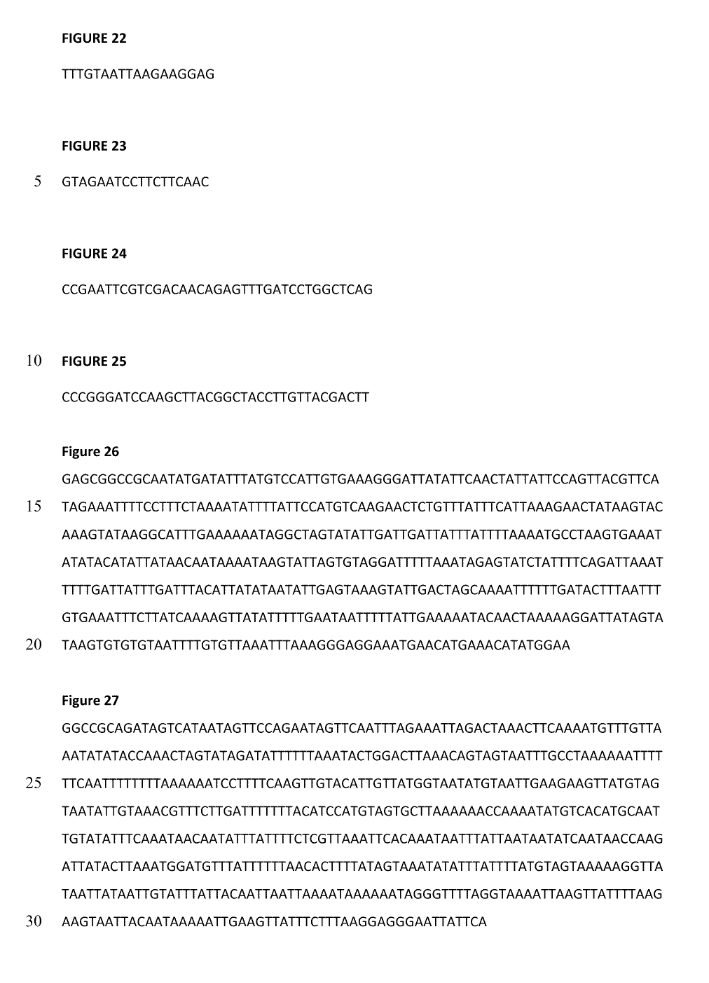

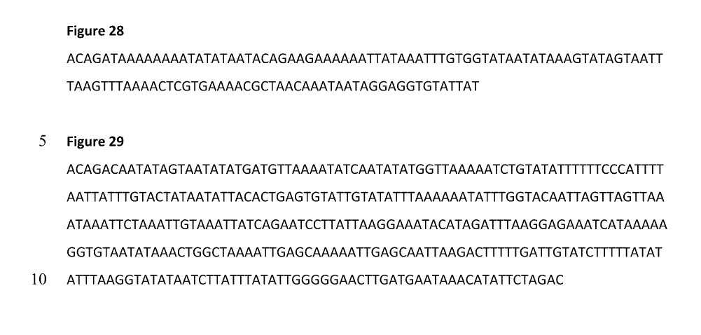

To verify the DNA transfer, a plasmid mini prep was performed from 10 ml culture volume using Zyppy plasmid miniprep kit (Zymo). PCR was performed with the isolated plasmid as template using primer pairs ermB-F (SEQ_ID NO. 20) plus ermB-R (SEQ_ID NO. 21), and SOE-GroESL-a-NdeI (SEQ_ID NO. 6) and SOE-GroESL-d-EcoRI (SEQ_ID NO. 9) to confirm the presence of the plasmid (FIG. 7). PCR was carried out using iProof High Fidelity DNA Polymerase (Bio-Rad Labratories) and the following program: initial denaturation at 98° C. for 30 seconds, followed by 35 cycles of denaturation (98° C. for 10 seconds), annealing (55° C. for 30 seconds) and elongation (72° C. for 15-60 seconds), before a final extension step (72° C. for 10 minutes).

To confirm the identity of the clones, genomic DNA was isolated (see above) and a PCR was performed against the 16s rRNA gene using oligonucleotides fD1 (SEQ_ID NO. 22) and rP2 (SEQ_ID NO. 23) (Weisberg W A, Barns S M, Pelletier D A and Lane D J: 16S rDNA amplification for phylogenetic study. J Bacteriol 1991, 173: 697-703) and iNtRON Maximise Premix PCR kit (Intron Bio Technologies) with the following conditions: initial denaturation at 94° C. for 2 minutes, followed by 35 cycles of denaturation (94° C. for 20 seconds), annealing (55° C. for 20 seconds) and elongation (72° C. for 60 seconds), before a final extension step (72° C. for 5 minutes). Sequencing results confirmed 99.9% identity against the 16S rRNA gene of C. autoethanogenum (Y18178, GI:7271109)—(GenBank accession number, gene ID number).

Overexpression of GroESL Enhanced Ethanol Tolerance of C. autoethanogenum DSM23693:

To investigate whether overexpression of GroESL enhances ethanol tolerance of C. autoethanogenum DSM23693, both wild type (WT) and transformed strain carrying plasmid pMTL85246-GroESL were challenged with different concentrations of ethanol (FIG. 8).

Growth experiments in triplicates were carried out in 50 ml PETC media (Table 1) in serum bottles sealed with rubber stoppers and 30 psi steel mill gas (collected from New Zealand Steel site in Glenbrook, NZ; composition: 44% CO, 32% N₂, 22% CO₂, 2% H₂) in the headspace as sole energy and carbon source. Different amounts of anaerobized ethanol was added to the media prior to inoculation to achieve final ethanol concentrations of 15 g/L, 30 g/L, 45 g/L and 60 g/L (which was confirmed by HPLC). All cultures were inoculated to the same optical density using the same pre-culture for either wild-type or transformed strain. Changes in biomass were measured spectrophotometrically at 600 nm until growth ceased. The maximum biomass of each culture was compared with the unchallenged culture.

Cultures that overexpressed Heat shock protein/chaperonin complex GroESL were generally found to have an increased ethanol tolerance when compared to an unchallenged culture. While growth of the wildtype ceased after addition of 60 g/l ethanol completely, the strain overproducing GroESL was still able to grow. The wild-type culture showed only 0.39 doubling when challenged with 45 g/l ethanol and biomass even dropped when 60 g/l ethanol was added, while the culture overproducing GroESL doubled 2.14 and respectively 1.27 times when challenged with 45 and respectively 60 g/l ethanol.

While the wild-type of C. autoethanogenum shows no growth at ethanol concentrations greater 50 g/l or 5% in serum bottle experiments (FIGS. 1 and 8), the modified strain which overproduces Heat shock protein/chaperonin complex GroESL was surprisingly able to grow even in presence of 60 g/l or 6% ethanol.

The invention has been described herein, with reference to certain preferred embodiments, in order to enable the reader to practice the invention without undue experimentation. However, a person having ordinary skill in the art will readily recognise that many of the components and parameters may be varied or modified to a certain extent or substituted for known equivalents without departing from the scope of the invention. It should be appreciated that such modifications and equivalents are herein incorporated as if individually set forth. Titles, headings, or the like are provided to enhance the reader's comprehension of this document, and should not be read as limiting the scope of the present invention.

The entire disclosures of all applications, patents and publications, cited above and below, if any, are hereby incorporated by reference. However, the reference to any applications, patents and publications in this specification is not, and should not be taken as, an acknowledgment or any form of suggestion that they constitute valid prior art or form part of the common general knowledge in any country in the world.

Throughout this specification and any claims which follow, unless the context requires otherwise, the words “comprise”, “comprising” and the like, are to be construed in an inclusive sense as opposed to an exclusive sense, that is to say, in the sense of “including, but not limited to”.

1-52. (canceled)

53. The recombinant microorganism capable of producing one or more products by fermentation of a substrate comprising CO, wherein the microorganism has an increased tolerance to ethanol compared to a parental microorganism.

54. The recombinant microorganism of claim 53, wherein the microorganism is tolerant of ethanol concentrations of at least approximately 5.5% by weight of fermentation broth (i.e. 55 g ethanol/L of fermentation broth).

55. The recombinant microorganism of claim 53, wherein the microorganism is adapted to express or over-express one or more enzymes adapted to increase tolerance to ethanol.

56. The recombinant microorganism of claim 55, wherein the one or more enzymes are chosen from the group consisting of stress proteins and chaperones.

57. The recombinant microorganism of claim 56, wherein the one or more enzymes are chosen from the group consisting of protein disaggregation chaperone (ClpB), class III stress response-related ATPase (ClpC), ATP-dependent serine protease (ClpP), Hsp70 chaperon (DnaK), Hsp40 chaperon (DnaJ), transcription elongation factor (GreA), Cpn10 chaperonin (GroES), Cpn60 chaperonin (GroEL), heat shock protein (GrpE), heat shock protein (Hsp18), heat shock protein (Hsp90), membrane bound serine protease (HtrA), methionine aminopeptidase (Map), protein chain elongation factor (TufA), protein chain elongation factor (TufB), Arginine kinase related enzyme (YacI), and functionally equivalent variants of any one or more thereof.

58. The recombinant microorganism of claim 57, wherein the one or more enzymes are GroES and GroEL.

59. The recombinant microorganism of claim 56, wherein the microorganism comprises one or more exogenous nucleic acids adapted to increase expression of one or more nucleic acids native to the microorganism and which encode the one or more enzymes.

60. The recombinant microorganism of claim 56, wherein the microorganism comprises one or more exogenous nucleic acids encoding and adapted to express the one or more enzymes.

61. The recombinant microorganism of claim 60, wherein the microorganism comprises one or more exogenous nucleic acids encoding each of GroES and GroEL.

62. The recombinant microorganism of claim 53, wherein the microorganism is selected from the group of acetogenic bacteria.

63. The recombinant microorganism of claim 62, wherein the microorganism is selected from the group consisting of Clostridium autoethanogenum, Clostridium ljungdahlii, Clostridium ragsdalei, Clostridium carboxidivorans, Clostridium drakei, Clostridium scatalogenes, Butyribacterium limosum, Acetobacterium woodii, Blautia producta, Eubacterium limosum, Moorella thermoacetica, Moorella thermautotrophica, Oxobacter pfennigii, and Thermoanaerobacter kiuvi.

64. The recombinant microorganism of claim 63, wherein the microorganism is Clostridium autoethanogenum DSM23693.

65. A fermentation method comprising fermenting a substrate comprising CO using a recombinant microorganism of claim 53, wherein the fermentation results in a product that includes at least ethanol.

66. The method of claim 65, further comprising:
(a) providing a substrate that includes CO to a bioreactor containing a culture of one or more microorganism of claim 53; and
(b) anaerobically fermenting the culture in the bioreactor to produce one or more products including ethanol.

67. The method of claim 65, wherein the ethanol concentration in the fermentation broth is at least approximately 5.5% by weight.

68. The method of claim 65, wherein the substrate comprises an industrial waste gas.

69. The method of claim 65, wherein the substrate comprises at least about 20% to about 100% CO by volume.

70. The method of claim 65, wherein the method reduces the total atmospheric carbon emissions from an industrial process.

71. The method of claim 65, wherein the method produces one or more other products.
